(19) United States
(12) Patent Application Publication
(10) Pub. No.: US 2024/0225602 A1
(43) Pub. Date: Jul. 11, 2024

(54) MULTI-DIMENSIONAL & MULTI-FREQUENCY ULTRASOUND TRANSDUCERS

(71) Applicant: FUJIFILM SONOSITE, INC., Bothell, WA (US)

(72) Inventors: Jimin Zhang, Bellevue, WA (US); Wei (Grace) Li, Bothell, WA (US); Ken Best, Bothell, WA (US); Keith Williams, Bothell, WA (US); Thomas Endres, Bothell, WA (US)

(21) Appl. No.: 18/613,694

(22) Filed: Mar. 22, 2024

Related U.S. Application Data
(63) Continuation-in-part of application No. 17/561,313, filed on Dec. 23, 2021.

(51) Int. Cl. A61B 8/00; G01S 7/52; G01S 15/89

(52) U.S. Cl. CPC A61B 8/4494; A61B 8/4488; A61B 8/467; A61B 8/54; G01S 7/52098; G01S 15/8925; G01S 15/8952

(57) ABSTRACT

Ultrasound devices that include and/or use a multi-dimensional and multi-frequency arrays of transducer elements for use with ultrasound and methods for using the same are disclosed. In some embodiments, an ultrasound device includes: an array having a center row of transducer elements of a first width that operate at a first frequency and two or more outer rows of transducer elements of a second or other widths that operate at a second or other frequencies different than the first frequency. A controller is configured to control the center row of transducer elements and two or more outer rows of transducer elements to operate at a same time or at different times.

### RELATED APPLICATION

The present application is continuation-in-part and claims the benefit of U.S. patent application Ser. No. 17/561,313, filed Dec. 23, 2021, and entitled “ARRAY ARCHITECTURE AND INTERCONNECTION FOR TRANSDUCERS”, which is incorporated by reference in its entirety.

### FIELD

Embodiments disclosed herein relate to ultrasound systems. More specifically, embodiments disclosed herein are related to ultrasound devices that include and/or use a multi-dimensional and multi-frequency array of transducer elements for use with ultrasound.

### BACKGROUND

In conventional ultrasound diagnostic imaging system applications, several separate ultrasound transducers (also referred to as ultrasound probes or ultrasound scanners) are used with the imaging system to accomplish various clinical needs for human body imaging. For example, a phased array is used for cardiac related diagnostics, a high frequency linear array for nerve visualization, and a curved linear array for abdominal imaging.

Because transducers are designed for specific applications, it is common for a clinician to switch between different types of ultrasound transducers to get images with optimized resolution and penetration depth during the imaging usages. However, switching transducers during the imaging diagnostics could interrupt clinical workflow, increase clinical exam time, and increase cleaning time of the transducers.

Another approach used by some manufacturers is to put two different functional arrays at the two separate ends of an ultrasound scanner. For example, in one implementation, a linear array could be mounted at one end of the scanner while a curved array is mounted at the opposite end of the scanner. While this approach may work for a wireless scanner, it will be difficult to manage the cable when a wired transducer is used. Further, switching the two ends of the scanner back and forth could bring some difficulties on workflow or require more time for array cleaning especially when clean clinical environments are required.

Thus, the approaches mentioned above either are too expensive to develop or not easy to use and clean in clinical environments.

### SUMMARY

Ultrasound devices that include and/or use a multi-dimensional and multi-frequency arrays of transducer elements for use with ultrasound and methods for using the same are disclosed. In some embodiments, an ultrasound device includes: a lens; an array coupled to the lens and having a center row of transducer elements of a first width that operate at a first frequency and two or more outer rows of transducer elements of a second or other widths that operate at a second or other frequencies different than the first frequency, where the first width is different than the second width and the center row is between the two or more outer rows. The ultrasound device also includes a controller coupled to the array and configured to control the center row of transducer elements and two or more outer rows of transducer elements to operate at a same time or at different times.

In some embodiments, an ultrasound device comprising: a lens; a high frequency array comprising a center row of transducer elements that operate at a first frequency and a low frequency array comprising two outer rows of transducer elements that operate at a second or other frequencies different than the first frequency, the center row of transducer elements being between the two outer rows of transducer elements. The ultrasound device also includes a controller coupled and configured to control each of the high frequency array and the low frequency array in a plurality of modes to operate at a same time or at different times. In some embodiments, the modes include a first mode in which the controller controls the high frequency array to operate as a linear array while low frequency array is not operating; a second mode in which the controller controls the low frequency array to operate as a phased array while the high frequency array is not operating; and a third mode in which the controller controls the high and low frequency arrays to operate at the same time to obtain signals for performing super broadband harmonic imaging.

In some embodiments, a method of controlling an ultrasound system includes controlling a multi-frequency and multi-dimensional array of transducer elements having a high frequency array comprising a center row of transducer elements that operate at a first frequency and a low frequency array comprising two outer rows of transducer elements that operate at a second or other frequencies different than the first frequency, where the center row of transducer elements is between the two outer rows of transducer elements. In some embodiments, controlling the multi-frequency and multi-dimensional array includes controlling both the high frequency array and low frequency array to operate at the same time; receiving, by both the high and low frequency arrays, reflected signals; and performing super broadband harmonic imaging based on the received signals.

### BRIEF DESCRIPTION OF THE DRAWINGS

The appended drawings illustrate examples and are, therefore, exemplary embodiments and not considered to be limiting in scope.

FIG. 1 illustrates some embodiments of an ultrasound system (e.g., an ultraportable ultrasound system) in an environment during an ultrasound examination.

FIG. 2 illustrates an ultraportable ultrasound system according with some embodiments.

FIGS. 3A-3D illustrate some embodiments of a front view of a multi-functional array transducer.

FIGS. 4A and 4B illustrate some embodiments of a transducer having two rows of transducer elements and five rows of transducer elements, respectively.

FIGS. 5A and 5B illustrate some embodiments of transducer element stacks that can be used to form arrays of transducer elements.

FIG. 6 illustrates an electrical diagram of some embodiments of a multi-functional transducer and interface to an imaging system.

FIG. 7 illustrates a sample of operation frequencies and their corresponding broader bandwidths of a multi-functional transducer having a triple row configuration.

FIGS. 8A and 8B illustrate samples of elevation beam patterns of a multi-functional transducer with a triple row configuration in accordance with some embodiments.

FIG. 9 illustrates samples of imaging modes created by using some embodiments of a multi-functional transducer with an imaging system.

FIGS. 10A-F illustrates some embodiments of example methods for controlling an ultrasound system.

FIG. 11 illustrates a block diagram of an example computing device that can perform one or more of the operations described herein, in accordance with some embodiments.

FIG. 12 illustrates an environment for an ultrasound system in accordance with some embodiments.

FIG. 13 illustrates an example user interface for an ultrasound system in accordance with some embodiments.

FIGS. 14A-C illustrate some embodiments of multi-dimensional array architectures.

FIG. 15 illustrates some embodiments of multi-dimensional array architectures.

### DETAILED DESCRIPTION

In the following description, numerous details are set forth to provide a more thorough explanation of the present invention. It will be apparent, however, to one skilled in the art, that the present invention may be practiced without these specific details. In other instances, well-known structures and devices are shown in block diagram form, rather than in detail, in order to avoid obscuring the present invention.

Embodiments disclosed herein include several array architectures and interconnection schemes for multi-functional and multi-frequency ultrasound transducers. Embodiments disclosed herein also include array-to-system electrical connection methods and several unique imaging modes for using the multi-functional and multi-frequency transducers with the imaging system.

In some embodiments, the ultrasound transducer has multiple functionalities (phased and linear), multiple elevation focal depths, multiple operation frequencies, super broader bandwidth that could provide clinicians a powerful tool for full-body scans without having to exchange transducers during the clinical procedures. The super broader bandwidths can be the result of an aggregation, or combining, of bandwidths (e.g., overlapping bandwidths, etc.) or resonances from multiple sub-arrays of transducer elements in a multi-functional transducer. Also, in some embodiments, the multi-functional transducer achieves broad bandwidths while maintaining or increasing sensitivity.

When part of a wired or wireless handheld ultrasound scanner, the multi-function transducers described herein are capable of being used for full body scanning, that will have huge advantages in various environments. In clinical applications, when multiple transducers with different operation frequencies are required, a single multi-functional transducer such as disclosed herein can provide detailed images with multiple operation frequencies in clinical diagnostics.

### EXAMPLE ULTRASOUND SYSTEMS

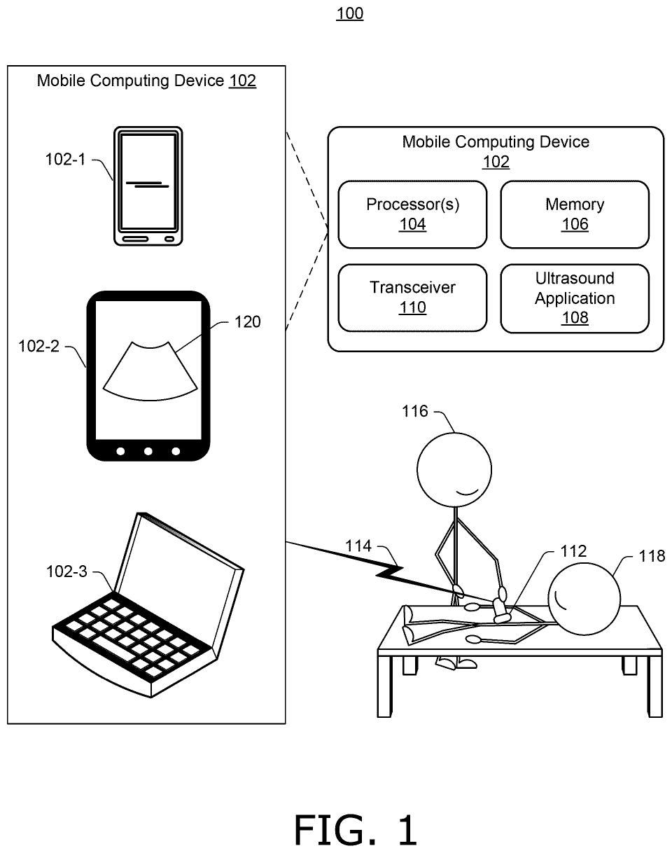

FIG. 1 illustrates some embodiments of an ultrasound system (e.g., an ultraportable ultrasound system) in an environment 100 during an ultrasound examination. The ultrasound system includes a mobile computing device 102. The mobile computing device 102 can include any suitable mobile computing device, with the illustrated examples including a smartphone 102-1, a tablet 102-2, and a laptop 102-3 (collectively referred to as the mobile computing device 102), which can also be referred to as a handset. The mobile computing device 102 includes hardware, software, and firmware for performing the ultrasound examination, such as one or more processors 104 and one or more memories 106. In some embodiments, the one or more memories 106 store instructions that when executed by the processor(s) 104 implement an ultrasound application 108 for performing the ultrasound examination, including controlling the ultrasound scanner 112 and viewing ultrasound images (e.g., the ultrasound image 120). The mobile computing device 102 also includes a transceiver 110 that can be used to communicate over a communication link 114 with the ultrasound scanner 112. In some embodiments, the communication link 114 includes a wireless communication link so that the mobile computing device 102 and the ultrasound scanner 112 are wirelessly coupled, e.g., paired. Additionally or alternatively, the communication link 114 can include a wired communication link that includes one or more cables.

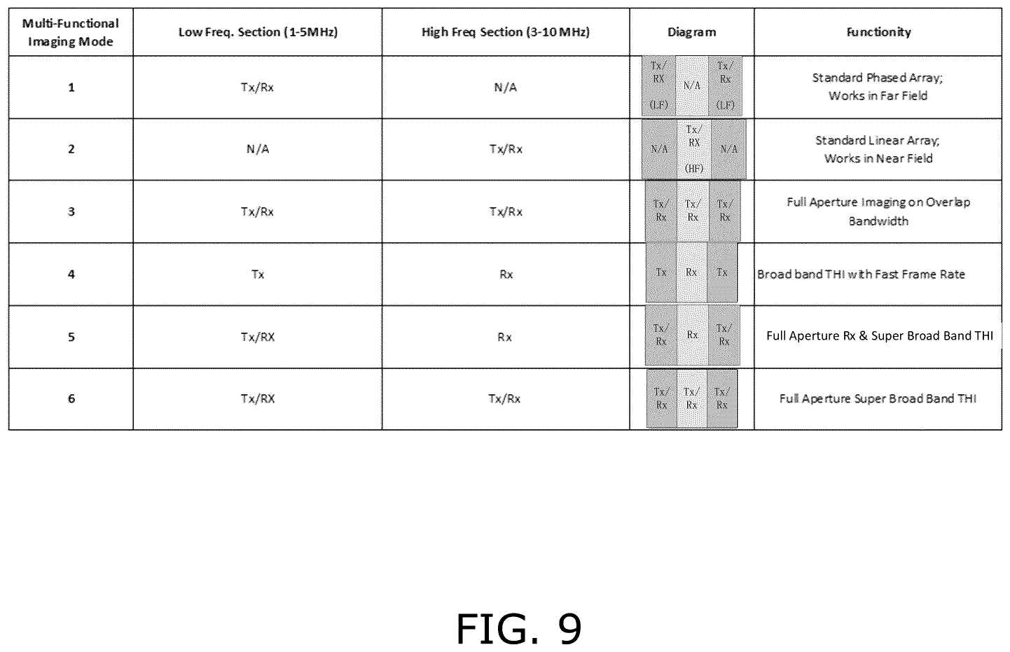

The ultrasound system 100 also includes the ultrasound scanner 112, which can be referred to as an ultrasound probe, ultrasound transducer as disclosed in more detail below, and the like. In some embodiments, the ultrasound scanner 112 is operated by an operator 116 (e.g., clinician, nurse, sonographer, etc.,) to transmit ultrasound at an anatomy of a patient 118 and receive reflections of the ultrasound from the patient anatomy as part of the ultrasound examination. The ultrasound system 100 can generate the ultrasound image 120 based on the reflections, and the computing device 102 can display the ultrasound image 120. In examples, the ultrasound scanner 112 is a multi-functional array transducer that includes multiple arrays, and the ultrasound system 100 can implement full aperture imaging, tissue harmonic imaging, super broadband imaging, combinations thereof, and the like, as further described below (e.g., with respect to FIG. 9).

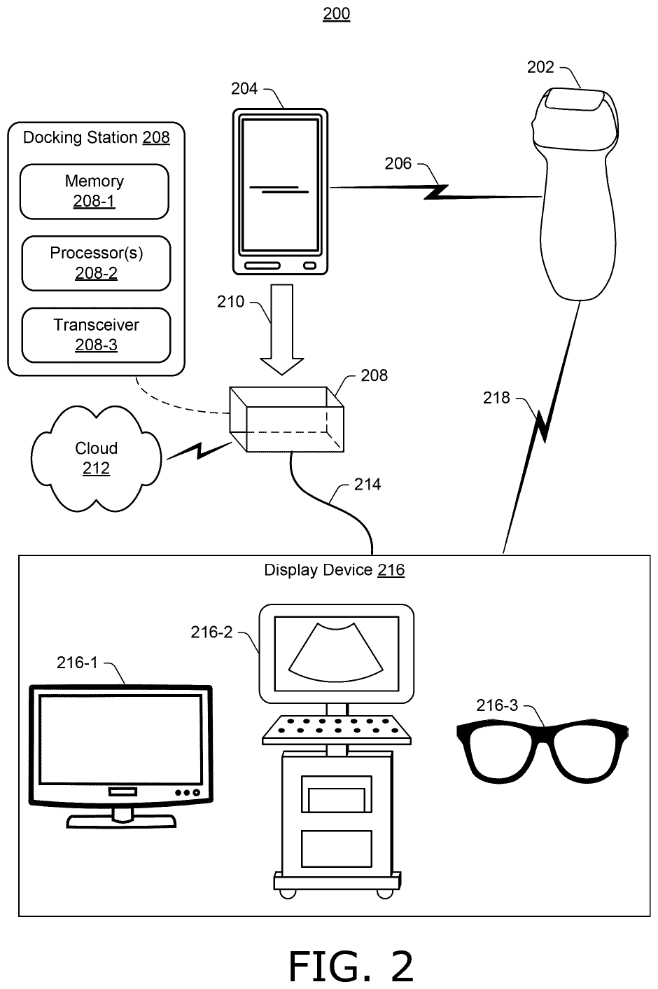

FIG. 2 illustrates some embodiments of an ultraportable ultrasound system 200. Referring to FIG. 2, the ultraportable ultrasound system 200 includes an ultrasound scanner 202 (which is an example of the ultrasound scanner 112 in FIG. 1) and a mobile computing device 204 (which is an example of the mobile computing device 102 in FIG. 1). The ultrasound system 200 is “ultraportable” in the sense that in some embodiments, it can be handheld and easily transported by a single user, unlike a larger cart-based or rack-based system. In some embodiments, the ultrasound scanner 202 can be used with an ultrasound system that is not ultraportable, such as a rack-mounted, stationary ultrasound system. The ultrasound scanner 202 and the mobile computing device 204 are paired with each other, and are thus coupled via the communication link 206 (which is an example of the communication link 114 in FIG. 1). In some embodiments, the communication link 206 includes a wireless communication link. Additionally or alternatively, the communication link 206 can include a wired communication link, including one or more cables.

The ultraportable ultrasound system 200 also includes a docking station 208 that can be configured to support (e.g., physically hold in place) the mobile computing device 204 when the mobile computing device 204 is inserted into the docking station 208. This insertion is illustrated by the arrow 210. The docking station 208 can be of any suitable form factor to support the mobile computing device 204, and is illustrated as a plain rectangular box in FIG. 2 for clarity. In some embodiments, the docking station 208 provides power to the mobile computing device 204 when it is docked (e.g., inserted into the docking station 208). Further, the docking station 208 can provide additional ultrasound resources to the ultraportable ultrasound system 200 when the mobile computing device 204 is supported by the docking station 208 (e.g., the mobile computing device 204 is docked). The additional ultrasound resources may not be available to the ultraportable ultrasound system 200 when the mobile computing device 204 is unsupported by the docking station 208 (e.g., it is not docked). The additional ultrasound resources can include, for example, but not limited to, advanced measurement capabilities (e.g., for cardiac parameters such as ejection fraction), implementations of machine-learned models (e.g. neural networks), advanced imaging modes (e.g., Doppler modes), and the like. In some embodiments, the additional ultrasound resources include controlling of one or more arrays of the ultrasound scanner 202. For instance, the additional resources can include the enabling of tissue harmonic imaging, full-aperture operation (with a bandwidth that covers the bandwidths of multiple arrays of the ultrasound scanner 202, combinations thereof, and the like. These modes of operation are described below in more detail.

Hence, the docking station 208 can include any suitable hardware, software, and/or firmware to provide the additional ultrasound resources, including memory 208-1 and processors 208-2 that can execute instructions stored by the memory 208-1 to provide the additional ultrasound resources. The docking station 208 also includes a transceiver 208-3 for communicating with a cloud 212. Cloud 212 can include any network, network resources, server, database, and the like for providing resources, including the additional ultrasound resources, to the ultraportable ultrasound system 200. In some embodiments, the cloud 212 is maintained by a care facility (e.g., hospital, clinic, etc.) where the ultraportable ultrasound system 200 is used to perform ultrasound examinations.

In some embodiments, the docking station 208 is included as part of the mobile computing device 204. Hence, the mobile computing device 204 can include the memory 208-1, the processors 208-2, and the transceiver 208-3. In some embodiments, the docking station 208 can be attached to a patient bed, so that the mobile computing device 204 can be placed in a stationary position relative to the patient bed during an ultrasound examination. Additionally or alternatively, the docking station can be attached to an ultrasound cart to hold the mobile computing device 204 during transport and/or during an ultrasound examination.

The docking station 208 is coupled via a communication link 214 to a display device 216 of the ultraportable ultrasound system 200. The transceiver 208-3 can facilitate communication between the docking station 208 and the display device 216 over the communication link 214. The communication link 214 can include a wired connection (e.g., a cable) to propagate data between the docking station 208 and the display device 216. The display device 216 can include any suitable device for displaying ultrasound data, illustrated examples of which include a monitor 216-1, an ultrasound machine 216-2, and smart glasses 216-3. These examples of the display device 216 are exemplary and meant to be non-limiting. In embodiments, the display device 216, e.g., the glasses 216-3, display ultrasound data (e.g., an ultrasound image) in an augmented reality (AR) or a virtual reality (VR) environment. In some embodiments, the additional ultrasound resources described above are provided to the ultraportable ultrasound system 200 by the display device 216.

In some embodiments, the mobile computing device 204 is configured to display a first user interface for controlling the ultraportable ultrasound system 200 when supported by the docking station 208 and a second user interface for controlling the ultraportable ultrasound system 200 when the mobile computing device 204 is unsupported by the docking station 208. For instance, the second user interface can include only basic ultrasound controls, such as controls for B-mode and M-mode imaging, and the first user interface can include advanced ultrasound imaging controls, such as controls for color Doppler, power Doppler, spectral Doppler imaging, tissue harmonic imaging, full-aperture imaging (described below in more detail), and the like. In some embodiments, a user can transfer components of a user interface displayed on the display device 216 to a user interface displayed by the mobile computing device 204 (and vice versa). For instance, a user can select a portion of a user interface on the display device 216, such as by drawing a boundary container around the portion to select the portion, and then perform a swipe gesture to an edge of the display device 216 to transfer the portion of the user interface to the mobile computing device 204. Additionally or alternatively, the user can transfer components of a user interface displayed on the mobile computing device 204 to a user interface displayed by the display device 216, such as with a selection trace and a swiping gesture.

In some embodiments, when the mobile computing device 204 is supported by the docking station 208, the ultraportable ultrasound system 200 can disable the communication link 206 between the mobile computing device 204 and the ultrasound scanner 202, and enable a communication link 218 between the ultrasound scanner 202 and the display device 216. Hence, the ultrasound scanner 202 can be paired with the display device 216.

In some embodiments, the ultraportable ultrasound system 200 facilitates the simultaneous use of multiple ultrasound scanners for use by different clinicians (not shown in FIG. 2 for clarity). For example, a first operator can operate a first ultrasound scanner that is coupled to the mobile computing device 204, a second operator can operate a second ultrasound scanner that is coupled to the docking station 208, and a third operator can operate a third ultrasound scanner that is coupled to the display device 216. Additionally or alternatively, one or more of the mobile computing device 204, the docking station 208, and the display device 216 can be simultaneously paired with multiple ultrasound scanners that are simultaneously operated by different operators. The display device 216 can display ultrasound data generated based on one or more of the ultrasound scanners. For instance, the smart glasses 216 can depict an AR or VR environment that overlays the data generated from two or more ultrasound scanners.

By providing the additional ultrasound resources to the ultraportable ultrasound system 200, e.g., from the cloud 212, the docking station 208, and/or the display device 216, the components of the ultraportable ultrasound system 200, including the mobile computing device 204 and the ultrasound scanner 202, can remain small in form factor and light weight, allowing them to be used for their intended purpose of ultra-portability. Further, the mobile computing device 204 and the ultrasound scanner 202 can consume less power than if they were required to implement the additional ultrasound resources. Hence, the ultrasound scanner 202 generates less heat, and thus can be used for longer scan times with shorter wait times between scans, resulting in better patient care. In some embodiments, the docking station 208 can be removably attached to the ultrasound scanner 202. Hence, the docking station 208 and the ultrasound scanner 202 can be transported as a single unit, to make it less susceptible to loss and/or theft. At the point of care, the docking station 208 can be quickly removed from the ultrasound scanner 202 for use.

### ARRAY ARCHITECTURE EXAMPLES

In some embodiments, a multi-functional array transducer is multi-dimensional and multi-frequency. In some embodiments, the multi-functional array transducer has a center row (sub-array) of transducer elements (e.g., PZT elements) that operate at one frequency and two outer rows (sub-arrays) of additional (e.g., low frequency) transducer elements (e.g., PZT elements) that operate at another frequency that is lower than the operating frequency of the high frequency transducer elements. In some embodiments, the two outer rows of low frequency transducer elements are side by side the center row of high frequency transducer elements with the center row being between the two outer rows. Furthermore, in some embodiments, the width of the transducer elements is different among the rows of elements. For example, in some embodiments, the width of the transducer elements in the center row is smaller than the width of the transducer elements in the outer rows. In some embodiments, the multi-functional array transducer has transducer elements that have different element widths and operation frequency that are selected based on clinical applications for which the multi-functional array transducer is to be used. In this way, the multi-functional array transducer is configurable based on the clinical application that is to be performed at the time.

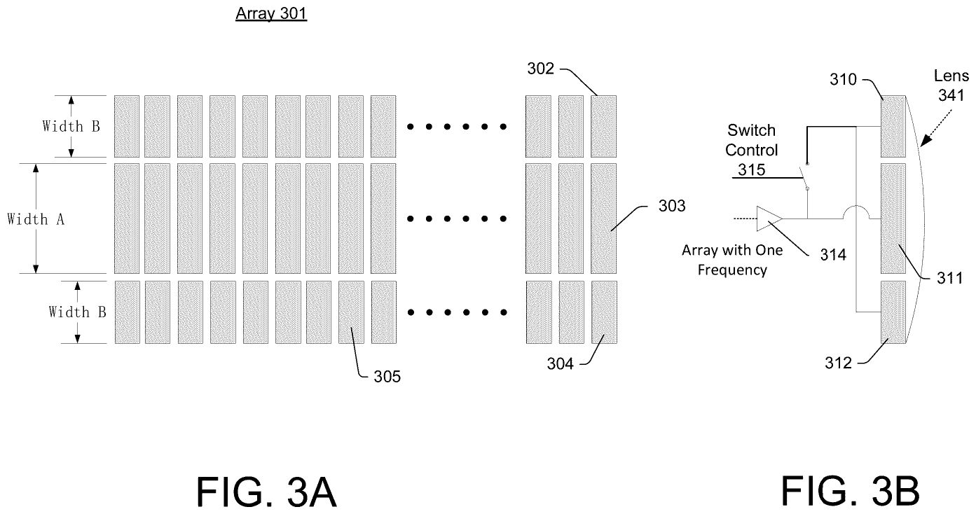

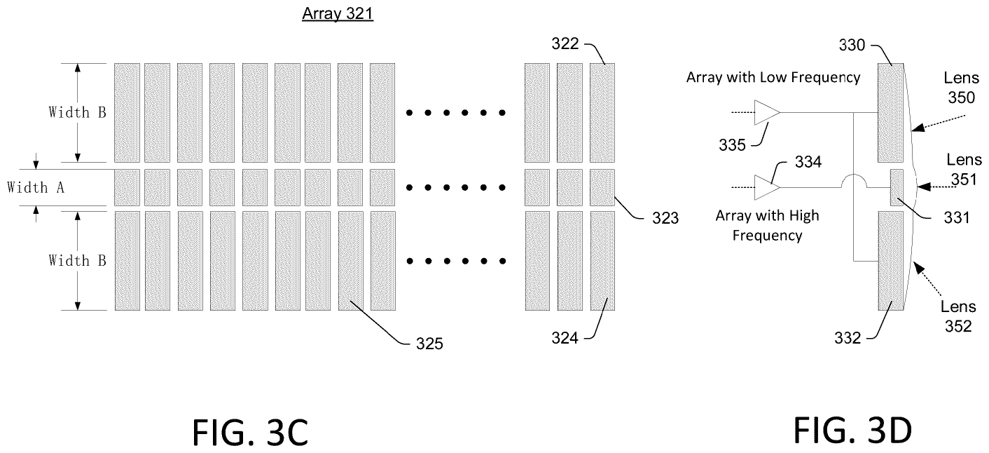

Some traditional multi-row array designs, such as the 1.25D, 1.5D or 1.75D, have the same sub-element area and operation frequency in each row of elements. FIGS. 3A-3D illustrate some embodiments of a front view of a multi-functional array transducer.

Referring to FIG. 3A, a 1.25D array transducer includes array 301 having rows 302-304 of transducer elements, such as, for example, transducer element 305 of row 304. Row 303 is the center row with rows 302 and 304 on opposite sides of row 303. The width of the transducer elements in row 303 is twice the size of the width of the transducer elements in rows 302 and 304. Also, each row transducer element has the same operation frequency as shown in side view of FIG.

3B. Referring to FIG. 3B, transducer element 310 is from row 302, transducer element 311 is from row 303, and transducer element 312 is from row 304, with lens 341 covering transducer elements 310-312. Transducer element 311 is driven with one frequency using driver 314. Switch 315 is coupled to driver 314 and transducer elements 310 and 312 and is controlled by a controller (not shown for clarity). When switch 315 is closed, driver 314 drives transducer element 310 of row 302 and transducer element 312 of row 304 with the same frequency as transducer element 311; when switch 315 is open, only transducer element 311 is driven. Thus, the controller operates array 301 in one mode in which all the rows of transducer elements are used and in another mode in which only the center row of transducer elements is used.

In contrast to the 1.25D array transducer of FIG. 3A, in some embodiments, the multi-functional transducers disclosed herein not only have different element widths for the different rows of elements but also have rows that operate at different frequencies. In some embodiments, the widths and/or operation frequency are based on clinical applications from one transducer.

Referring to FIG. 3C, multi-functional transducer includes array 321 having rows 322-324 of transducer elements, such as, for example, transducer element 325 of row 324. Row 323 is the center row with rows 322 and 324 on opposite sides of row 323, thereby forming a symmetric arrangement. The width of the transducer elements in row 323 is less than the size of the width of the transducer elements in rows 322 and 324. Also, each transducer element of a given row has the same operation frequency, and the elements of rows 322 and 324 operate at a lower frequency than the elements of row 323, as shown in side view of FIG. 3D. Referring to FIG. 3D, transducer element 330 is from row 322, transducer element 331 is from row 323, and transducer element 332 is from row 324, with lens 351 covering transducer element 331 and lens 350 covering transducer elements 330 and 332. Note that, in some embodiments, the design in FIGS. 3C and 3D is for phased/linear combination in which the center row width is smaller than the width of other outside rows and where the higher frequency is two times of the low frequency array elements. However, the teachings herein are not limited to such configurations and can be expanded to configurations such as, for example, a linear/linear combination, a curved linear/linear combination, etc.

In some embodiments, transducer elements in row 323 including transducer element 331 are driven with one frequency using driver 334, while transducer elements in rows 322 and 324, along with transducer elements 330 and 332, are driven by driver 335 that are separate circuitry with different frequencies. In some embodiments, driver 335 drives rows 322 and 324 with a low frequency while driver 334 drives row 331 with a high frequency in comparison to the frequency that drives rows 322 and 324. Note that while rows 322 and 324 can be driven separately from row 323, all of rows 322-324 can be driven at the same time depending on which mode the array 321 is operating. A controller coupled to array 321 (not shown for clarity) causes drivers 334 and 335 to drive the transducer elements in rows 322-324 and is configured to control the center row of transducer elements and two or more outer rows of transducer elements with separate circuitry to operate at a same time or at different times.

Note that in FIGS. 3A-3D, the widths of the elements of each row in a multi-row configurations are not limited to Width A is less than Width B. In some clinical applications, the width of elements in the rows can be such that the Width A is greater than Width B, which can provide better acoustic or array functional performances. In some other embodiments, Width A is equal to Width B.

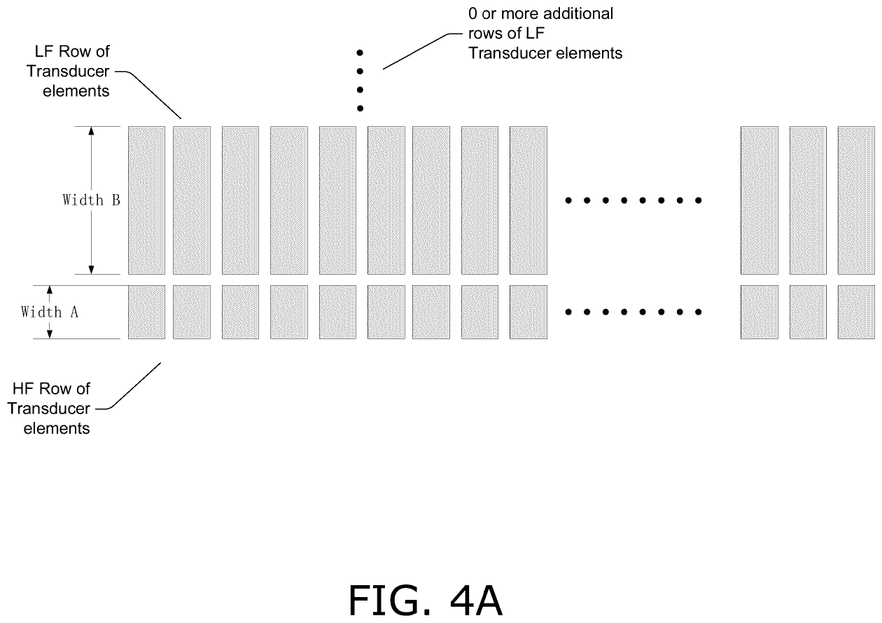

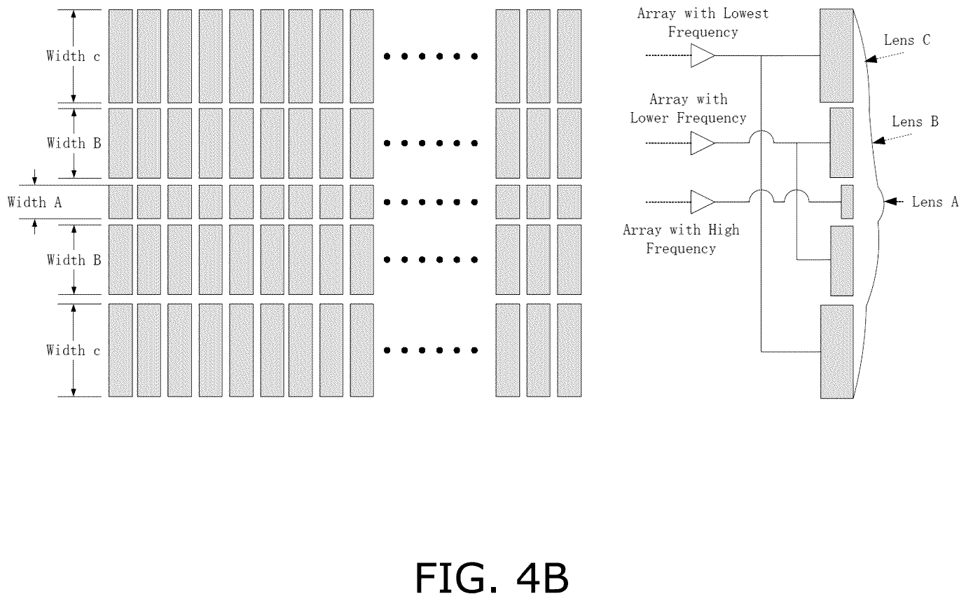

In some embodiments, the transducer is not limited to three rows of transducer elements. For example, in other symmetric designs, the transducer has five rows, such as shown in FIG. 4B. seven rows, nine rows, etc. In some embodiments, the transducer is not limited to being symmetric. That is, the transducer can be asymmetric and have a different number of rows of transducer element on one side of the center row of transducer elements than the other side. The number can be one or more additional rows of transducer elements on one side of the center row in comparison to the other side. For example, in some embodiments, the transducer only has one row of transducer elements next to the center row, such as shown in FIG. 4A, one or more additional rows on one side of the center row, such as shown as an example in FIG. 4B. Any number of additional rows can be included on one of the sides.

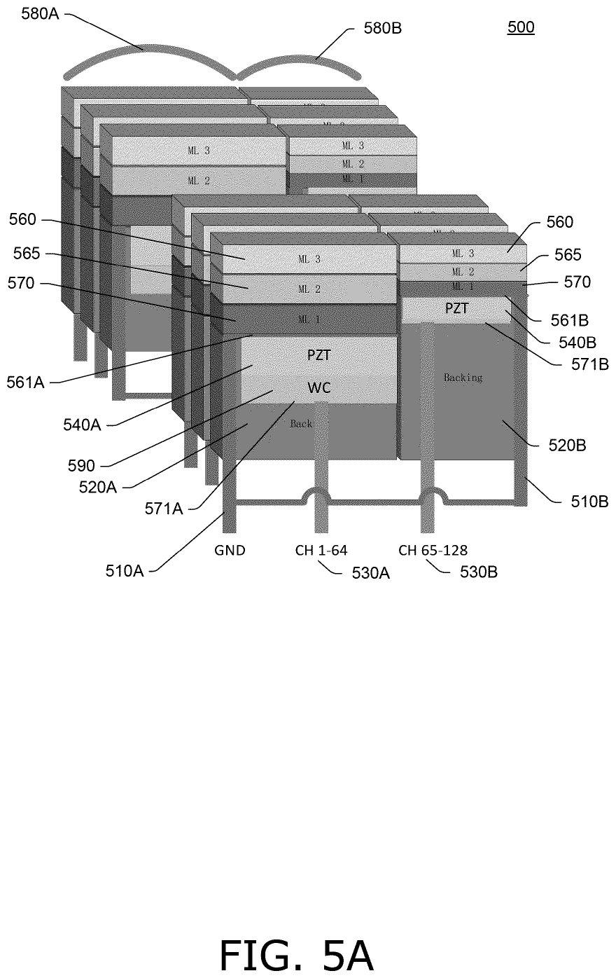

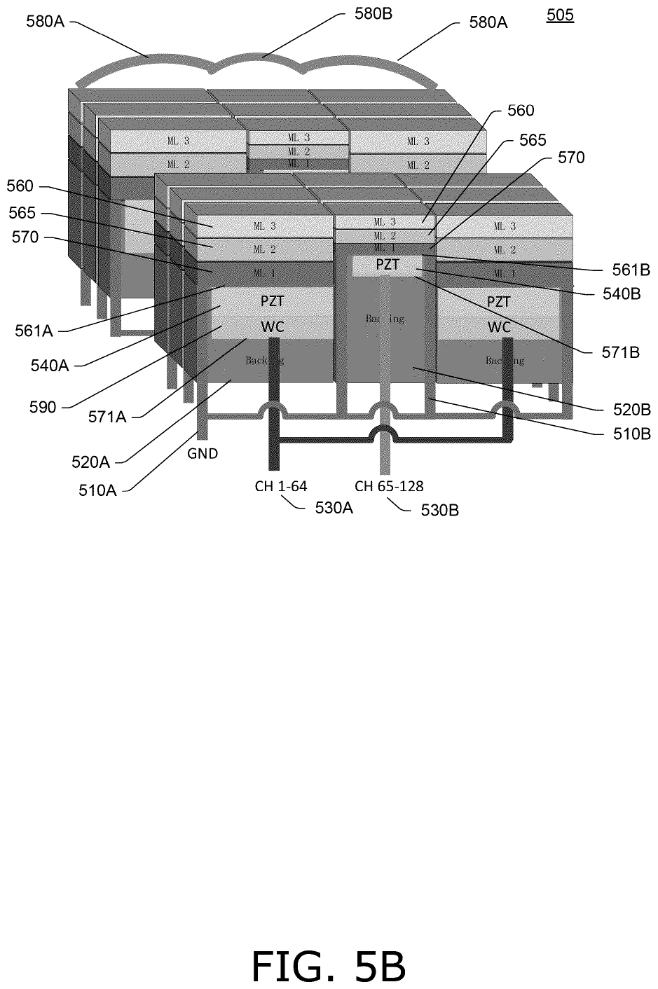

Some embodiments of the transducer provide a solution to an interconnection problem from electrical to acoustic elements in multi-row transducers by embedding multiple signals and/or grounds inside a backing block. FIGS. 5A and 5B illustrate elevational cross-section views of some embodiments of transducer element acoustic stacks (modules) that can be used to form arrays of transducer elements, such as shown, for example, in FIGS. 3A-3D. More specifically, FIG. 5A illustrates some embodiments of a portion of an array with two different operation frequency sections that are constructed in a double configuration, e.g., side by side, and FIG. 5B shows illustrates embodiments of a portion of an array with two different operation frequency sections that are constructed in a triple configuration, e.g., the center section with higher operation frequency and the two side sections with lower operation frequencies (e.g., FIG. 3C).

Referring to FIGS. 5A and 5B, the center row (e.g., FIG. 3C, row 323) and the outer rows (e.g., FIG. 3C, row 322, row 324) have very similar but not common acoustic stacks. In these embodiments, an electrical signal connecting each row is embedded inside a backing block. Ground wires are also embedded inside the backing block on the side shoulder section. Thus, as described in greater detail below, grounds from these multiple rows can be connected through a conductive matching layer or a matching layer with a conductive surface, or an electrode. The matching layer includes an electrode on an inside (acoustic) surface which faces the piezoelectric material, such as a piezoelectric ceramic (e.g., PZT, etc.).

FIG. 5A illustrates an elevation cross-section view of a multi-row transducer 500 with a double (two) row configuration. Referring to FIG. 5A, the transducer 500 includes examples of two acoustic stacks, with the stack on the left representing a low frequency acoustic stack and the stack on the right representing a high frequency stack, e.g., the high frequency stack can operate at a higher operational frequency than the low frequency stack. Each acoustic stack includes a backing block, such as backing block 520A and 520B. In the two acoustic stacks, two grounds 510A and 510B are electrically coupled to matching layer 1 and embedded in backing block. Electrical signals 530A and 530B are embedded in and traverse the backing blocks 520A and 520B, respectively. In some embodiments, signals 530A and 530B as well as grounds 510A and 510B can be coupled to flex circuits or other well-known interconnection technology to connect to a circuit board (e.g., PCB) in the enclosure housing the multi-row transducer.

Each stack of the multi-row transducer 500 also includes three matching layers 560, 565, and 570. The first matching layer 560 includes a conductive surface that includes an electrode 561. In some embodiments, electrodes 561A and 561B are electrically connected to grounds 510A and 510B. In some embodiments, grounds 510A and 510B are coupled together, e.g., electrically connected.

Stacks of transducer 500 also includes an acoustic layer, such as piezoelectric layer 540 (e.g., PZT), below the conductive surface that includes the electrode 561. The matching layers 560, 565, 570 are located between the piezoelectric layer 540 and acoustic lens 580. In some embodiments, the lens 580 depicted in FIG. 5A is a single radius of curvature (ROC) focus lens, but some other embodiments can include other types of lenses, such as, for example, a multi-ROC focus lens.

Each stack also includes an electrode 571 electrically connected to signals 530, which can include and/or represent one of channels 1-64 and 65-128. In some embodiments, the low frequency stack also includes a Tungsten carbide (WC) layer interfacing electrode 590 to signal lines 530. In some embodiments, the high frequency stack also includes a Tungsten carbide (WC) layer interfacing its electrode to signal lines. In some other embodiments, such a layer is not included in either the low or high frequency stacks. Alternatively, this layer can be tungsten (W) or other suitable materials with higher acoustic impedance than PZT.

FIG. 5B illustrates an elevation cross-section view of a multi-row transducer 505 with a triple (three) row configuration. Referring to FIG. 5B, the transducer 505 includes examples of three acoustic stacks, with the acoustic stacks on the left and right representing the low frequency acoustic stacks and the acoustic stack in the center representing a high frequency acoustic stack. For example, the high frequency stack can operate at a higher ultrasound frequency that the low frequency stack. The signals from the transducer elements for both rows of low frequency acoustic stacks are combined to provide channels 1-64, and transducer elements for the center row combine to provide channels 65-128. Note that the channel mapping set forth above and herein are merely examples, and other transducer configurations can have different channel mappings.

The acoustic stacks inside multi-function transducer could have different operation frequencies, element geometries, focal depths, and beam characteristics, such as beam width and lens focal depth. The acoustic stacks of FIGS. 5A and 5B include an acoustic lens in front of the transducers and in some embodiments, the lenses 580A and 580B could have different radius of curvatures to provide different focal depths to improve the acoustic performances on different clinical applications.

As discussed above with respect to symmetric and non-symmetric row configurations, the acoustic stacks can be built with a non-symmetric format with a combination of two, three, four, five, or more separated acoustic stacks operating at different operational frequencies (e.g., ultrasound frequencies) and with different imaging modes such as phased array, or linear array. In some embodiments, two or more of these sub-arrays have different imaging planes. In some other embodiments, all these sub-arrays have different imaging planes.

The acoustic stacks can also be built with a symmetric format with the combination of a number of 1+2n (n=1, 2, 3, . . . ) sub-arrays, e.g., three, five, seven, or more separated acoustic stacks operating at different operations frequencies and with different imaging modes (e.g., a phased array, a linear array, etc.). With the exception of the center sub-array, the symmetric sub-arrays on both sides of the center could have the same operation frequency. However, the sub-arrays with a different distance to the center can have different operation frequencies. In some embodiments, all of the sub-arrays have the same imaging plane in the middle of the transducer.

Thus, embodiments of multi-row transducers are disclosed that provide unique acoustic designs and architectures to integrate multiple acoustic stacks with different functionalities into one array enclosure. In some embodiments, the array enclosure has similar dimensions as traditional one-dimensional arrays used in diagnostic ultrasound imaging applications.

### Unique Acoustic Designs to Simplify Manufacturing Process

Techniques are disclosed herein include manufacturing processes to build cost-effective, high performance, multi-row transducers. In some embodiments, for example, in FIGS. 5A and 5B, the acoustic designs enable multi-functional arrays with phased and linear arrays together, and the phased array sections can operate at a lower frequency f₀ while the linear array section can operate at a higher frequency, such as, for example, 2f₀. In some embodiments, the phased array has a ½ wavelength element pitch at operation frequency f₀, i.e., phase array pitch equals λ/2 which equals ½·V/f₀, while the linear array section has 1 wavelength element pitch at operation frequency 2f₀, i.e., linear array pitch equals V/(2·f₀) which equals ½·V/f₀. In some embodiments, the element pitches for both the phased and linear arrays are the same (½·V/f₀). However, the phased array can operate at f₀, which is half of the operation frequency of the linear array (2f₀). The lower frequencies phased array has deeper penetration but the higher frequency linear array provides better resolution in near field in this multi-functional transducer.

Note that the techniques disclosed herein are not limited to λ/2. In some embodiments, based on the operation frequencies of the phased and linear arrays, a different pitch can be warranted (e.g., 0.8 λ, etc.).

The combination of phased and linear arrays with f₀ and 2f₀ as operation frequencies provide a simplification in the manufacturing process. For example, the multi-functional array could be diced at the same time with the same dicing pitch, which could greatly simplify the element dicing while keeping all the sections of the elements aligned well.

In FIGS. 5A and 5B, the acoustic designs of multi-functional array can also include two phased arrays or linear arrays together. The sub-arrays could have different operation frequencies, but with the same pitch. For example, a high frequency sub-linear array can have an approximately 1 wavelength pitch, Δd, and the same distance, Δd, can be used for a lower frequency sub-linear array with element pitch less than 1 wavelength, providing better angle responses for the lower frequency sub-linear array. In other words, the multi-functional transducer design has freedom with respect to the element pitch among the sub-arrays.

In some embodiments, the multi-functional transducer is built by combining each individual sub-arrays together. The combining can be performed by gluing them together. The combining can be performed by assembling sub-arrays together through mechanical structures. In some embodiments, each individual sub-array is built separately.

### Interconnection of Multi-functional Transducers with Imaging Systems

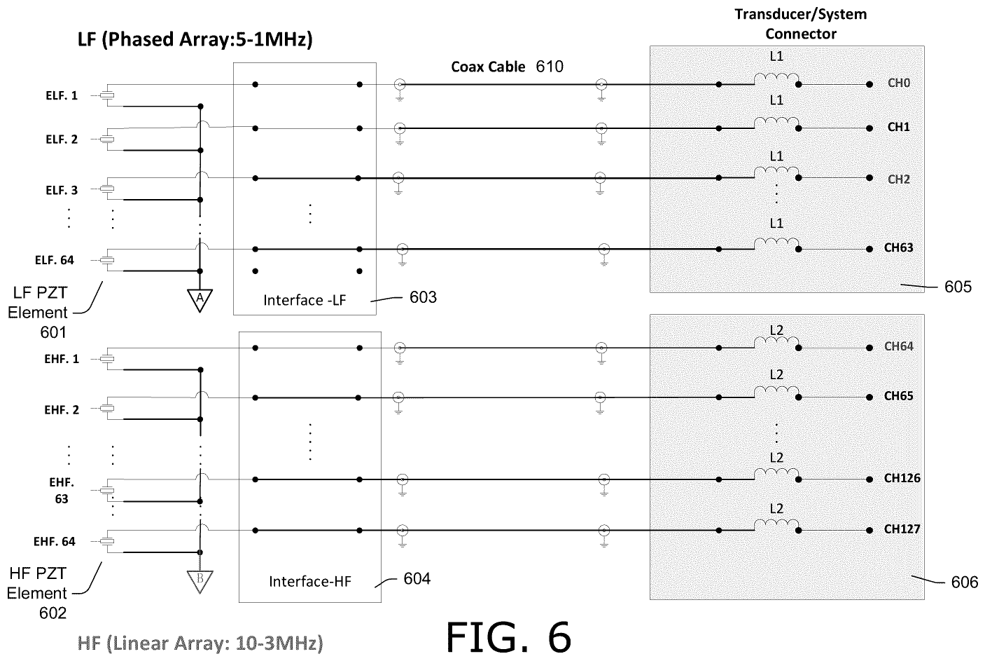

FIG. 6 illustrates an electrical diagram of some embodiments as an example of the electrical interconnection of the multi-functional transducer with an imaging system. Referring to FIG. 6, the transducer includes low frequency array (e.g., a phased array section) and a high frequency array (e.g., a linear array section). In some embodiments, the phase array operates from 1-5 MHz, while the linear array runs operates from 3-10 MHz.

In some embodiments, the phased array section with 64 transducer elements 601 (e.g., PZT, etc.) operating at a lower frequency (ELF) are connected to imaging system channels 1-64 via an interface board 603, coax cables 610, and a transducer/system connector 605 containing tuning inductors of a first value L1. The linear array section with 64 transducer elements 602 (e.g., a piezo material (e.g., PZT), etc.) operating at a higher frequency (EHF) than the ELF are connected to imaging system channels 65-128 via an interface board 604, coax cables 610, and a transducer/system connector 606 containing tuning inductors of a second value L2, which is different from the inductor value of connector 605.

In some embodiments, the elements of lower frequency and higher frequency arrays (ELF and EHF) have not only the different acoustic architectures and designs, such as PZT and matching layer thickness, but also are tuned with different inductor values to obtain the best overall performances. Note that the size (e.g., values) of the tuning inductors can be different depending on the operating frequency of the transducer elements.

### Acoustic Performance with Multi-functional Transducer

In some embodiments, the multi-functional array transducer provides unique flexibility and an acoustic method to create ultra-broadband operation frequencies and bandwidth, narrow beam widths and image resolutions in near and far field, and deeper penetration depths to meet various clinical needs in a single transducer. More specifically, when operated together at the same time, the bandwidths of each of the sub-arrays of transducer elements in the multi-functional array transducer are combined and, in essence, to extend the overall bandwidth, thereby creating an ultra-broadband bandwidth.

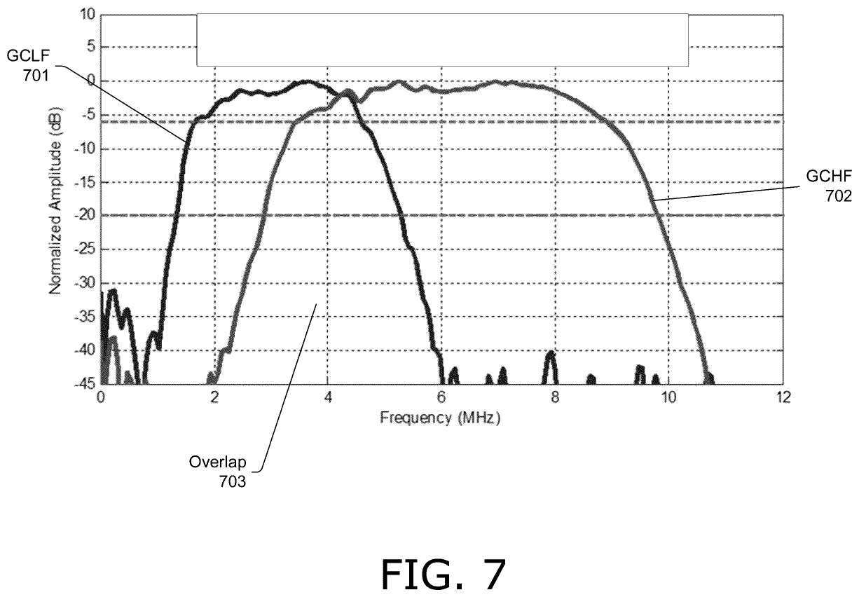

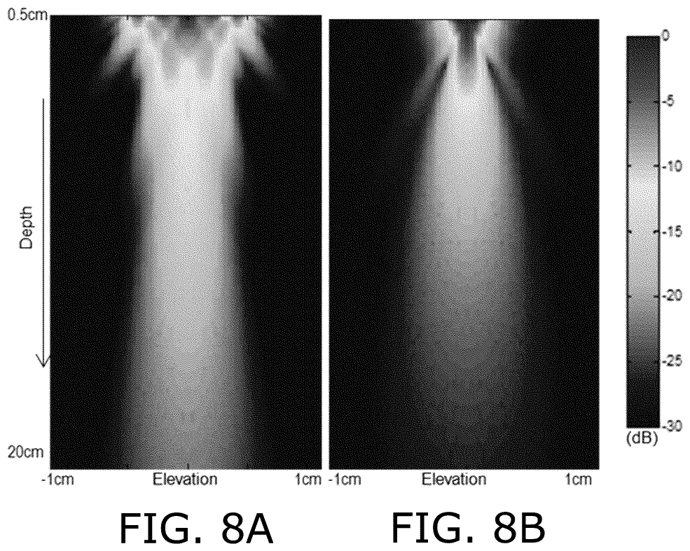

FIG. 7 illustrates operation frequencies and bandwidths of some embodiments of a multi-function transducer with a triple row (sub-array) configuration (e.g., FIG. 3C). Referring to FIG. 7, the bandwidth of the low frequency rows of transducer elements operating as a phased array is shown as graph 701 and bandwidth of the high frequency rows of transducer elements operating as a linear array is shown as graph 702. By combining both low and high frequencies and their broader bandwidth, the multi-functional transducer covers an operation frequency range from approximately 1 MHz to 10 MHz for −20 dB bandwidth.

By incorporating a unique lens design method, such as, for example, multi-radius (e.g., multi-ROC) acoustic lens in front of the high and low frequency sections, with the multi-function transducer's elevation section widths, the multi-functional transducer's elevation beam pattern design can be further improved in both near and far fields, compared to a single-ROC lens. For example, for the elevation of the triple row configuration depicted FIG. 5B, FIGS. 8A & 8B show beam patterns for the low frequency phased array and the high frequency linear array, respectively.

In some embodiments, the multi-radius lens for different acoustic sections can also reduce the attenuation of the ultrasound energy passing through the lens. In clinical applications, in Figure FIGS. 8A & 8B, when imaging the near field within 5 cm from the transducer front surface, the high frequency transducer sub-array with narrow beam is used (FIG. 8B), while the low frequency transducer sub-array on the two sides of the high frequency transducer sub-array is used when a need for imaging objects farther than 5 cm (FIG. 8A). The low frequency beam width has a wider beam width from within 5 cm and converged beyond 5 cm. However, the low frequency is used beyond 5 cm while high frequency covers the usages within 5 cm. In this way, clinical needs of high resolution in the near field is covered by the high frequency sub-array (e.g., a linear array) and the needs of penetration in the far field is covered by the low frequency sub-array (e.g., a phased array). In operation, both near and far fields can be imaged without changing a grip orientation on the ultrasound probe, unlike conventional ultrasound probes.

### Operational Modes Using a Multi-Functional Transducer

The multi-functional transducer can provide a great flexibility for the ultrasound imaging system to control ultrasound resolution and penetration to meet various clinical needs. In some embodiments, the multi-functional transducer can be configured to use one or more of at least six unique imaging modes with the imaging system. Some of these imaging modes results in an expanded bandwidth when using both high and low frequency sections of the multi-functional transducer at the same time. FIG. 9 illustrates the six imaging modes. Note that there can be other modes and/or an ultrasound system does not have to include all of Modes 1-6 described above. Furthermore, in some embodiments, during these modes, the low frequency section of the transducer operates with a frequency range from 1-5 MHz and the high frequency section of the transducer operates with a frequency range from 3-10 MHz. However, in some other embodiments, the low and high frequency sections operate with different operating frequency ranges. The frequency ranges described here exemplary and not meant to be limiting.

In clinical applications, the multi-functional transducer can switch between the imaging modes without changing the transducers, or in some cases, without even changing the operator's grip on the transducer/probe. The control of the switch between the imaging modes can use, but not limited to, a push button at the transducer, a pressure sensor at the transducer (e.g., grip equipped pressure to sense mode selection by a user, etc.), a push button at the system, a switch at the system or the transducer, a voice control command, a touch screen, an IMU with haptic feedback, etc. In one example, the ultrasound system can automatically and without user intervention switch between imaging modes based one or more of an anatomy being imaged (e.g., as determined by a machine-learned model), a current and/or subsequent step of an ultrasound protocol, such as the extended focused assessment with sonography in trauma (EFAST) protocol, an amount of battery life left (or charge remaining, or scan time) for the ultrasound probe, and a pressure applied from the probe to the patient.

Referring to FIG. 9, Mode 1 is where the low frequency section of the multi-functional transducer is used as a transmitter of ultrasound and a receiver of ultrasound reflections, while the high frequency section of the multi-functional transducer is not used. For example, in the case of the multi-functional transducer having a triple configuration (e.g., FIG. 3C), the outer rows (sub-arrays) are used as a transmitter of ultrasound and a receiver of ultrasound reflections, while the center row (sub-array) is not used. When operating in this manner, the low frequency section of the multi-functional transducer can be used as a standard phased array and works in the deeper depths.

Mode 2 is where the high frequency section of the multi-functional transducer is used as a transmitter of ultrasound and a receiver of ultrasound reflections, while the low frequency section of the multi-functional transducer is not used. For example, in the case of the multi-functional transducer having a triple configuration (e.g., FIG. 3C), the outer rows (sub-arrays) are not used, while the center row (sub-array) is used as a transmitter of ultrasound and a receiver of ultrasound reflections. When operating in this manner, high frequency section of the multi-functional transducer can be used as a standard linear array and works in the shallower depths.

In some embodiments, full-body scans can be performed by using different functional sections of the transducer (e.g., Mode 1 and Mode 2). The low frequency section can perform as a phased array with deep penetration in clinical applications, while the high frequency array can perform as a linear array with high resolution in clinical applications.

Mode 3 can be used to have the multi-functional transducer operate as a conventional array transducer with a full aperture, i.e. both sections operated together to produce the overlapped section of the frequency bandwidth (e.g., overlap 703 where graphs 701 and 702 overlap in FIG. 7). In this case, the multi-functional transducer can be controlled to obtain a desired level of sensitivity by adding the bandwidths of the two together to obtain the overlapped section with increased sensitivity. In some embodiments, the amount of overlap can be controlled by selecting operation frequencies of the high frequency section (e.g., the center row (sub-array), etc.) and low frequency section (e.g., the outer rows (sub-arrays), etc.). More specifically, in Mode 3, the high frequency section of the multi-functional transducer is used as a transmitter of ultrasound and a receiver of ultrasound reflections, and the low frequency section of the multi-functional transducer is used as a transmitter of ultrasound and a receiver of ultrasound reflections. For example, in the case of the multi-functional transducer having a triple configuration (e.g., FIG. 3C), the outer rows (sub-arrays) is used as a transmitter of ultrasound and a receiver of ultrasound reflections, and the center row (sub-array) is used as a transmitter of ultrasound and a receiver of ultrasound reflections.

In Mode 4, the low frequency section of the multi-functional transducer is used as a transmitter, and the high frequency section of the multi-functional transducer is used as a receiver. For example, in the case of the multi-functional transducer having a triple configuration (e.g., FIG. 3C), the outer rows (sub-arrays) is used as a transmitter, and the center row (sub-array) is used as a receiver. Using this mode, a fast frame rate super broadband harmonic imaging (sbTHI) can be created. In some embodiments, sbTH is Faster than the Pulse-Inversion-THI (PI-THI), and it doubles the frame rate of PI-THI. In the traditional tissue harmonic imaging, the transducer is used as transmitter and receiver. Due to its limited bandwidth, a pulse inversion technique is used in the tissue harmonic imaging (THI), e.g., the transducer transmits two separate pulses with 180-degree phase differences, and the received reflected signals from the tissues are added together. The reflected signal from the initial transmitting signals in tissues are cancelled out due to their 180-degree phase differences, and the 2nd harmonic signals generated from the tissue non-linear effects are enhanced. When the multi-functional transducer is used in Mode 4, most of bandwidth of the high frequency array has no overlap with the low frequency array, and a broad band low frequency transmitting signal will be filtered out (e.g., suppressed) by the high frequency array. If the 2nd harmonic and even higher harmonics generated from the low frequency transmission signal in tissues are received in the no-overlap bandwidth of the high frequency array, the reflected signal from the low frequency transmitting signal are filtered out by the high frequency array itself.

Therefore, no pulse inversion is required to cancel out the transmitting signal, and a fast frame rate, single pulse broad band THI can be realized.

In some embodiments, Mode 4 can be used in a cardiac application when a high frame rate is required to image the fast heart beats. However, the standard pulse inversion THI technique can also use this Mode 4 when a high frame rate is not required.

Mode 5 is a further extension from Mode 4 and can be used to increase the receiving aperture for deeper tissue sbTHI. In Mode 5, both low and high frequency sections of the multi-functional transducer are used in receiving. The low frequency array uses the lower-half of its frequency-spectrum-bandwidth in transmission. Both the low frequency and the high frequency array are used as receivers. Note that either the pulse inversion sbTHI or a fast frame rate sbTHI can be used in Mode 5 depending on clinical application.

Mode 6 is similar to Modes 4 and 5. In Mode 6, both low and high frequency sections (sub-arrays) are used in transmission and receiving. For example, in the case of the multi-functional transducer having a triple configuration (e.g., FIG. 3C), the outer rows (sub-arrays) are used as a transmitter and a receiver, and the center row (sub-array) is used as a transmitter and receiver. In some embodiments, in this mode, the multi-functional transducer is used as a super broadband transducer that can use pulse inversion THI within a much broader bandwidth.

In any of Modes 1 to 6, the pulse inversion THI can be used when a high frame rate THI is not required.

### EXAMPLE FLOW DIAGRAMS

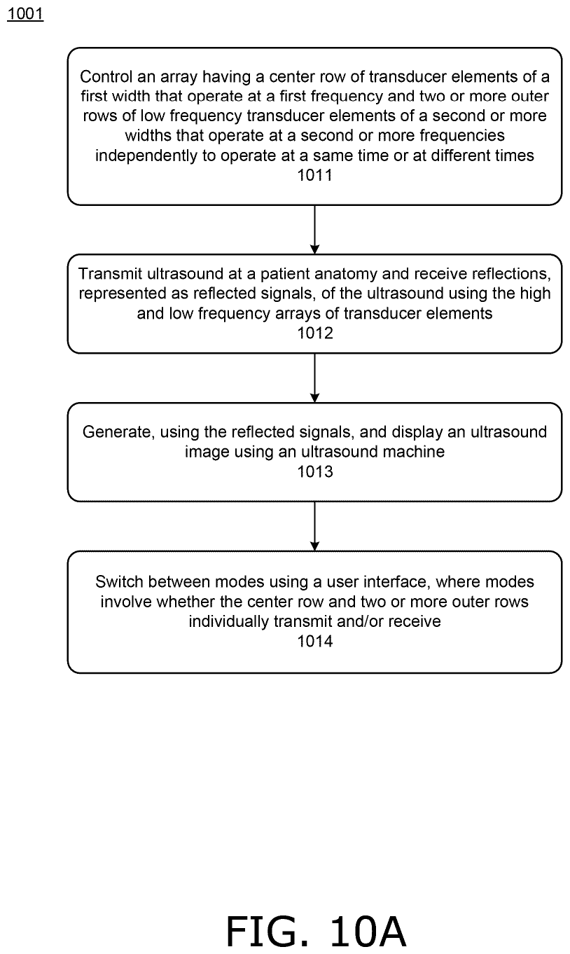

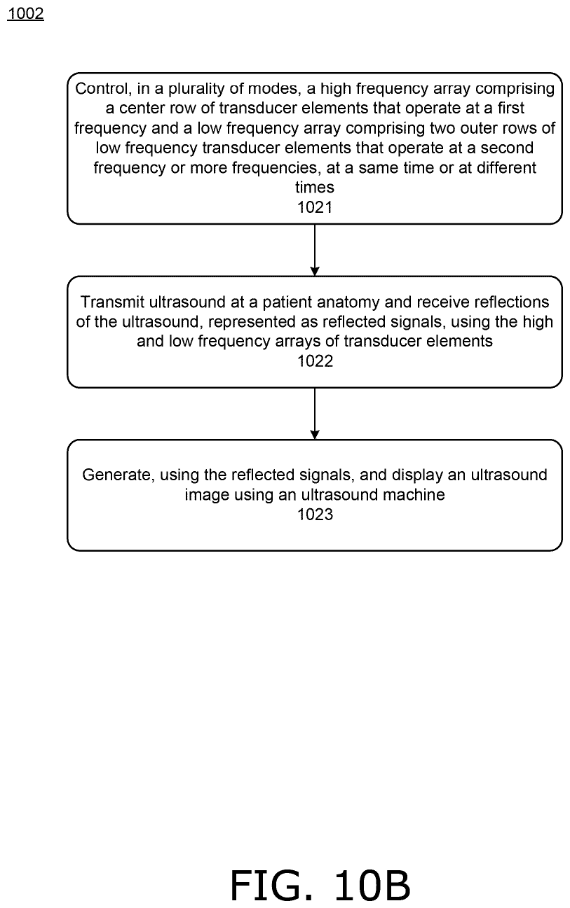

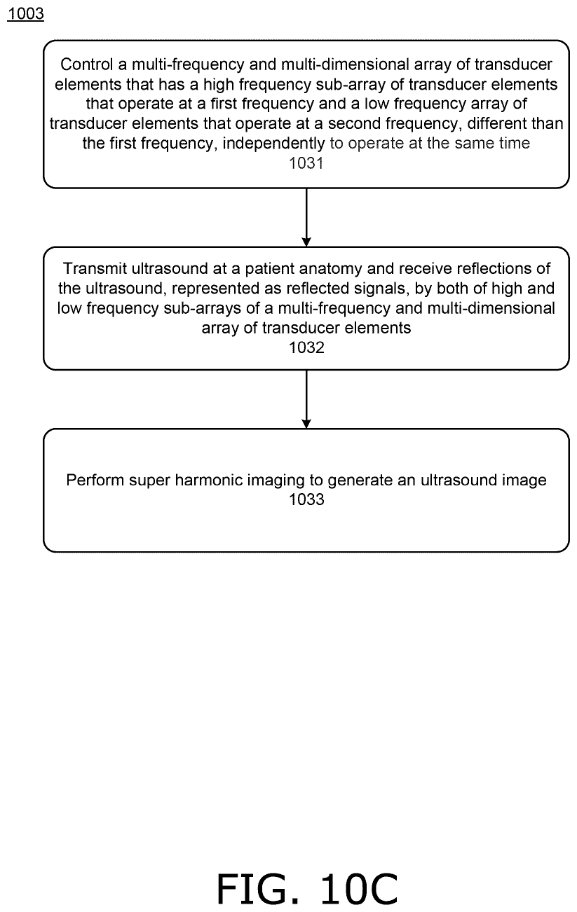

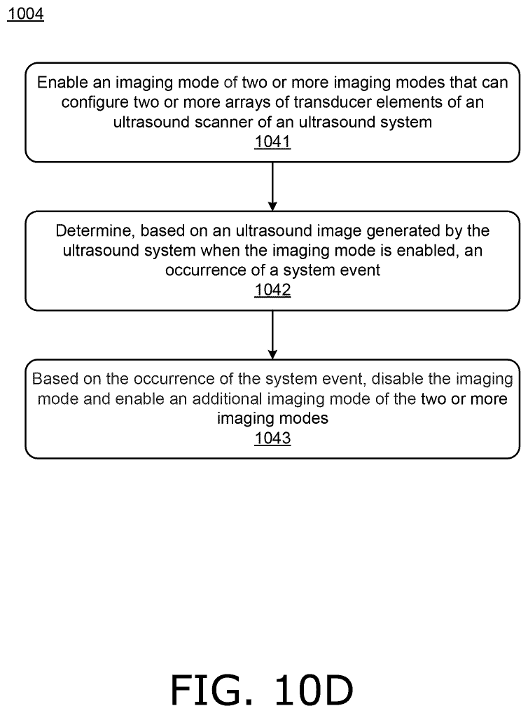

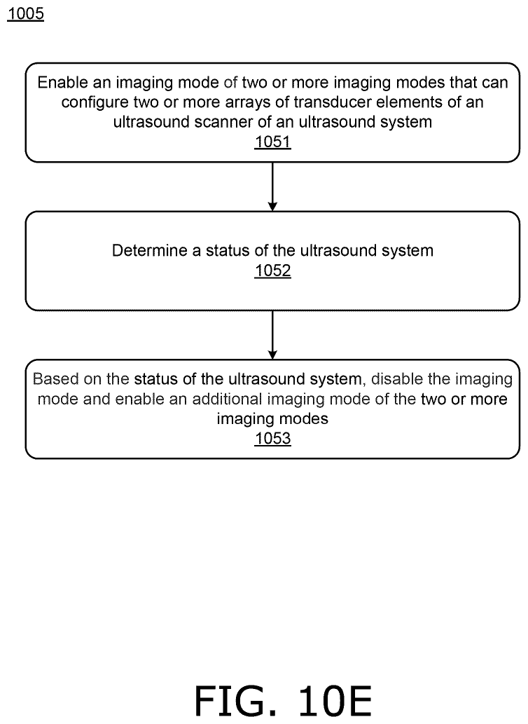

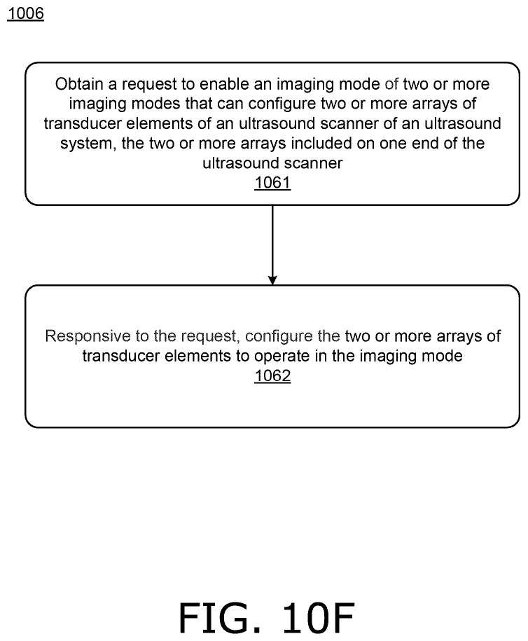

FIG. 10A illustrates some embodiments of an example method 1001 for controlling an ultrasound system. Operations of the method can be performed by processing logic that can comprise hardware (circuitry, dedicated logic, etc.), software (such as is run on a general-purpose computer system or a dedicated machine), firmware, or combinations thereof. The processing logic can be included in an ultrasound system. In some embodiments, the ultrasound system can include an ultrasound scanner, a mobile computing device (e.g., a handset), a display device (e.g., an ultrasound machine), a docking station, and a processor system. In some embodiments, the ultrasound system can include an ultrasound probe, a display device, and a processor system.

Referring to FIG. 10A, method 1001 includes controlling an array having a center row of transducer elements (e.g., PZT elements, etc.) of a first width that operate at a first frequency and two or more outer rows of low frequency transducer elements (e.g., PZT elements, etc.) of a second width that operate at a second frequency independently to operate at a same time or at different times (block 1011). In some embodiments, the second frequency is lower than the first frequency, the first width is smaller than the second width and the center row of transducer elements is between the two outer rows of transducer elements. In some embodiments, during operation, the center row operates as a linear array and a pair of rows of the two or more rows operates as a phased array. In some embodiments, the first frequency is twice the second frequency, and wherein element pitches for both high frequency array and the low frequency array are equal. In some embodiments, the high and low frequency arrays are part of a scanner or probe having a controller that controls the arrays to operate at a same time or at different times.

In some embodiments, the controller controls the array in a plurality of modes in which either the center row of transducer elements or the two or more rows of transducer elements is operating or both the center row of transducer elements and the two or more rows of transducer elements are operating at the same time based on which mode of the plurality of modes is being used. In some embodiments, the controller is configured to control the center row of transducer elements and two or more outer rows of transducer elements independently in one of the modes to operate at the same time to obtain signals for performing super broadband harmonic imaging by controlling the center row of transducer elements to perform a receive operation while controlling the two or more rows of transducer elements to perform transmit operations.

In some embodiments, the modes include one or more of a first mode in which the controller controls the high frequency array to operate as a linear array while low frequency array is not operating, a second mode in which the controller controls the low frequency array to operate as a phased array while the high frequency array is not operating, a third mode in which the controller controls the high and low frequency arrays to operate at the same time to obtain signals for performing super broadband harmonic imaging, a fourth mode in which the controller causes both the high and low frequency arrays to perform both transmit and receive operations to provide signals for full aperture imaging on an area of bandwidth produced by both that overlaps, a fifth mode in which the controller causes the high frequency array to perform a receive operation and the low frequency array to perform both transmit and receive operations to provide signals for THI, and a sixth mode in which the controller causes both the high and low frequency arrays to perform both transmit and receive operations to provide signals for super broadband full aperture THI. In some embodiments, during the third mode, the high frequency array filter out reflections from transmitted signals from the low frequency array.

Using the high and low frequency arrays of transducer elements, ultrasound is transmitted at a patient anatomy and receive reflections of the ultrasound by the high and low frequency arrays which are represented as reflected signals based on the mode (block 1012). In some embodiments, one or both arrays (e.g., center and outer rows of the transducer elements) transmit ultrasound and one or both arrays receive reflected signals.

In some embodiments, each of the transducer elements is part of an acoustic stack that includes a backing block through which at least a signal coupled to said each transducer element traverses. In some embodiments, each of the transducer elements is part of an acoustic stack, and stacks associated with transducer elements of the center row have different focal depths and beam characteristics than stacks associated with transducer elements of the two or more rows. In some embodiments, one or more system connectors interface signals from each of the transducer elements of the center row of transducer elements and the two or more rows of transducer elements to an ultrasound system, the one or more system connectors comprising first and second sets of tuning inductors, the first set of tuning inductors for interfacing signals from the transducer elements of the center row of transducer elements and the second set of tuning inductors for interfacing signals from the transducer elements of the two or more rows of transducer elements using tuning inductors with different inductor values than tuning inductors of the first set of tuning inductors.

Using the reflected signals, an ultrasound image is generated and displayed using an ultrasound machine (block 1013). In some embodiments, a computing device (e.g., an imaging subsystem) of the ultrasound system generates the image based on the received signals.

In some embodiments, the method also includes switching between modes using a user interface (e.g., one or more buttons, sensors, or switches coupled to a probe enclosure) (1014). Note that such as switch can be performed to change the imaging mode and/or when the user is going to image another type of anatomy.

FIG. 10B illustrates some embodiments of an example method 1002 for controlling an ultrasound system. Operations of the method can be performed by processing logic that can comprise hardware (circuitry, dedicated logic, etc.), software (such as is run on a general-purpose computer system or a dedicated machine), firmware, or combinations thereof. The processing logic can be included in an ultrasound system. In some embodiments, the ultrasound system can include an ultrasound scanner, a mobile computing device (e.g., a handset), a display device (e.g., an ultrasound machine), a docking station, and a processor system. In some embodiments, the ultrasound system can include an ultrasound probe, a display device, and a processor system.

Referring to FIG. 10B, method 1002 includes controlling a high frequency array comprising a center row of transducer elements (e.g., PZT elements, etc.) that operate at a first frequency and a low frequency array comprising two outer rows of low frequency transducer elements (e.g., PZT elements, etc.) independently in a plurality of modes to operate at a same time or at different times (block 1021). In some embodiments, the second frequency is lower than the first frequency, and the center row of transducer elements is between the two outer rows of transducer elements. In some embodiments, the first frequency is twice the second frequency, and wherein element pitches for both high frequency array and the low frequency array are equal. In some embodiments, the high and low frequency arrays are part of a scanner or probe having a controller that controls the arrays to operate at a same time or at different times.

In some embodiments, the modes include one or more of a first mode in which the controller controls the high frequency array to operate as a linear array while low frequency array is not operating, a second mode in which the controller controls the low frequency array to operate as a phased array while the high frequency array is not operating, a third mode in which the controller controls the high and low frequency arrays to operate at the same time to obtain signals for performing super broadband harmonic imaging, a fourth mode in which the controller causes both the high and low frequency arrays to perform both transmit and receive operations to provide signals for full aperture imaging on an area of bandwidth produced by both that overlaps, a fifth mode in which the controller causes the high frequency array to perform a receive operation and the low frequency array to perform both transmit and receive operations to provide signals for THI, and a sixth mode in which the controller causes both the high and low frequency arrays to perform both transmit and receive operations to provide signals for super broadband full aperture THI. In some embodiments, during the third mode, the high frequency array filter out reflections from transmitted signals from the low frequency array.

Using the high and low frequency arrays of transducer elements, ultrasound is transmitted at a patient anatomy and receive reflections of the ultrasound by the high and low frequency arrays which are represented as reflected signals based on the mode (block 1022). In some embodiments, depending on the mode, one or both arrays (e.g., center and outer rows of the transducer elements) transmit ultrasound and one or both arrays receive reflected signals.

Using the reflected signals, an ultrasound image is generated and displayed using an ultrasound machine (block 1023). In some embodiments, a computing device (e.g., an imaging subsystem) of the ultrasound system generates the image based on the received signals.

FIG. 10C illustrates some embodiments of an example method 1003 for controlling an ultrasound system. Operations of the method can be performed by processing logic that can comprise hardware (circuitry, dedicated logic, etc.), software (such as is run on a general-purpose computer system or a dedicated machine), firmware, or combinations thereof. The processing logic can be included in an ultrasound system. In some embodiments, the ultrasound system can include an ultrasound scanner, a mobile computing device (e.g., a handset), a display device (e.g., an ultrasound machine), a docking station, and a processor system. In some embodiments, the ultrasound system can include an ultrasound probe, a display device, and a processor system.

Referring to FIG. 10C, method 1003 includes controlling a multi-frequency and multi-dimensional array of transducer elements that has a high frequency sub-array of transducer elements (e.g., PZT elements, etc.) that operate at a first frequency and a low frequency array of transducer elements (e.g., PZT elements, etc.) that operate at a second frequency, different than the first frequency, independently to operate at the same time (block 1031). In some embodiments, the high frequency sub-array of transducer elements comprises a center row of transducer elements and a low frequency array comprising two outer rows of low frequency transducer elements, where the center row of transducer elements is between the two outer rows of transducer elements. In some embodiments, the multi-frequency and multi-dimensional array is part of a scanner or probe having a controller that controls the array to operate in this way.

Using the multi-frequency and multi-dimensional array of transducer elements, ultrasound is transmitted at a patient anatomy and receive reflections of the ultrasound by both of the high and low frequency sub-arrays which are represented as reflected signals (block 1032). In some embodiments, both sub-arrays (e.g., center and outer rows of the transducer elements) receive reflected signals. In some embodiments, the bandwidth of the received signals covers bandwidth across both the high and low frequency arrays.

Based on the received signals, super broadband harmonic imaging is performed (block 1033). In some embodiments, a computing device (e.g., an imaging subsystem) of the ultrasound system performs the super broadband harmonic imaging based on the received signals. The result of performing the super broadband harmonic imaging is the display of an image generated using an ultrasound machine.

FIG. 10D illustrates some embodiments of an example method 1004 for controlling an ultrasound system. Operations of the method can be performed by processing logic that can comprise hardware (circuitry, dedicated logic, etc.), software (such as is run on a general-purpose computer system or a dedicated machine), firmware, or combinations thereof. The processing logic can be included in an ultrasound system. In some embodiments, the ultrasound system can include an ultrasound scanner, a mobile computing device (e.g., a handset), a display device (e.g., an ultrasound machine), a docking station, and a processor system configured to implement a controller. In some embodiments, the ultrasound system can include an ultrasound probe, a display device, and a processor system.

Referring to FIG. 10D, method 1004 includes enabling an imaging mode of two or more imaging modes that can configure two or more arrays of transducer elements of an ultrasound scanner of an ultrasound system (block 1041). The method 1004 also includes determining, based on an ultrasound image generated by the ultrasound system when the imaging mode is enabled, an occurrence of a system event (block 1042). The method 1004 also includes, based on the occurrence of the system event, disabling the imaging mode and enabling an additional imaging mode of the two or more imaging modes (block 1043).

In some embodiments, the ultrasound system can determine the occurrence of the system event, disable the imaging mode, and enable the additional imaging mode without changing the ultrasound scanner. For instance, the same ultrasound scanner can be used for both the imaging mode and the additional imaging mode, without swapping the ultrasound scanner, rotating or moving the ultrasound scanner, or exchanging the ultrasound scanner for a different ultrasound scanner.

In some embodiments, the controller is implemented to, for at least one of the imaging mode and the additional imaging mode, operate at least two arrays of the two or more arrays of transducer elements. These arrays can operate with at least one of different bandwidths and different frequencies. In some embodiments, these arrays include a linear array and a phased array. Additionally or alternatively, these arrays can include a first linear array and a second linear array. Additionally or alternatively, these arrays can include a first curvilinear array and a second curvilinear array.

In some embodiments, the occurrence of the system event includes a determination that the ultrasound image includes a patient anatomy. In some embodiments, the ultrasound system includes a machine-learned model (e.g., a neural network) implemented to make the determination. Additionally or alternatively, the ultrasound system can select, the additional imaging mode based on the patient anatomy.

In some embodiments, the occurrence of the system event includes a determination that the ultrasound image is part of a protocol step, such as step of a FAST or EFAST ultrasound protocol. Additionally or alternatively, the occurrence of the system event can include a determination that the ultrasound image includes an interventional instrument. Additionally or alternatively, the occurrence of the system event can include a determination that the ultrasound image has an image quality score that is above or below a threshold score. The ultrasound system can include one or more machine-learned models implemented to make one or more of the determinations of the occurrences of the system events.

In some embodiments, at least one of the imaging modes and the additional imaging mode includes at least one of a full aperture imaging mode, a tissue harmonic imaging mode, and a mode that combines full aperture imaging and tissue harmonic imaging. In an example, the imaging mode and the additional imaging mode can have different frame rates.

In some embodiments, the ultrasound system includes a display device implemented to display a user interface configured to display a visual representation of at least one mode of the imaging mode and the additional imaging mode. The visual representation can indicate at least one of an ultrasound frequency, a bandwidth, a transmission path, a reception path, and the transducer elements enabled for at least one mode. For instance, the visual representation can include one or more of text, an icon, an image, a graphic, an animation, a number, an arrow, and the like to indicate an ultrasound frequency, a bandwidth, a transmission path, a reception path, and the transducer elements enabled for the at least one mode.

FIG. 10E illustrates some embodiments of an example method 1005 for controlling an ultrasound system. Operations of the method can be performed by processing logic that can comprise hardware (circuitry, dedicated logic, etc.), software (such as is run on a general-purpose computer system or a dedicated machine), firmware, or combinations thereof. The processing logic can be included in an ultrasound system. In some embodiments, the ultrasound system can include an ultrasound scanner, a mobile computing device (e.g., a handset), a display device (e.g., an ultrasound machine), a docking station, and a processor system configured to implement a controller. In some embodiments, the ultrasound system can include an ultrasound probe, a display device, and a processor system.

Referring to FIG. 10E, method 1005 includes enabling an imaging mode of two or more imaging modes that can configure two or more arrays of transducer elements of an ultrasound scanner of an ultrasound system (block 1051). The method 1005 also includes determining a status of the ultrasound system (block 1052). The method 1005 also includes, based on the status of the ultrasound system, disabling the imaging mode and enabling an additional imaging mode of the two or more imaging modes (block 1053).

In some embodiments, the status of the ultrasound system includes an amount of battery charge for a battery of the ultrasound scanner. Additionally or alternatively, the ultrasound system can include an ultrasound machine configured to generate an ultrasound image, and the status of the ultrasound system can include an amount of battery charge for a battery of the ultrasound machine. Additionally or alternatively, the status of the ultrasound system can include an amount of scan time remaining.

In some embodiments, the status of the ultrasound system includes a determination of whether the ultrasound scanner is touching a patient. The ultrasound scanner can include one or more pressure sensors, and the processor system can make the determination based on pressure data generated by the one or more pressure sensors. Additionally or alternatively, the status of the ultrasound system can indicate the ultrasound system is operating according to a protocol step when the imaging mode is enabled.

In some embodiments, the status of the ultrasound system can indicate a receipt of a user selection via a user interface, the user selection indicating the additional imaging mode. The ultrasound scanner can include the user interface. Additionally or alternatively, the ultrasound system can include an ultrasound machine configured to generate an ultrasound image, and the ultrasound machine can include the user interface that is implemented to display the ultrasound image. In some embodiments, the status of the ultrasound system indicates an activation of an examination preset. Additionally or alternatively, the status of the ultrasound system can indicate a selection of a gain or depth.

In some embodiments, the ultrasound scanner includes the two or more arrays of transducer elements on a same end of the ultrasound scanner. The two or more arrays of transducer elements can be included in an enclosure that can be removably attached to the ultrasound scanner.

FIG. 10F illustrates some embodiments of an example method 1006 for controlling an ultrasound system. Operations of the method can be performed by processing logic that can comprise hardware (circuitry, dedicated logic, etc.), software (such as is run on a general-purpose computer system or a dedicated machine), firmware, or combinations thereof. The processing logic can be included in an ultrasound system. In some embodiments, the ultrasound system can include an ultrasound scanner, a mobile computing device (e.g., a handset), a display device (e.g., an ultrasound machine), a docking station, and a processor system configured to implement a controller. In some embodiments, the ultrasound system can include an ultrasound probe, a display device, and a processor system.

Referring to FIG. 10F, method 1006 includes obtaining a request to enable an imaging mode of two or more imaging modes that can configure two or more arrays of transducer elements of an ultrasound scanner of an ultrasound system, the two or more arrays included on one end of the ultrasound scanner (block 1061). The method 1006 also includes configuring, responsive to the request, the two or more arrays of transducer elements to operate in the imaging mode (block 1062).

In some embodiments, the two or more arrays are contained in a common enclosure that is attached to the one end of the ultrasound scanner. In some embodiments, the enclosure can be removed from the ultrasound scanner and reattached to the ultrasound scanner

In some embodiments, the ultrasound system includes a display device implemented to display a user interface. The user interface can receive the request as a user selection.

In some embodiments, the ultrasound system includes a machine-learned model implemented to generate an inference based on an ultrasound image generated by the ultrasound system. The ultrasound system can generate the request based on the inference. The inference can include at least one of a label, a classification, a segmentation, an object identification, an additional image, and a probability.

In some embodiments, the ultrasound scanner includes one or more pressure sensors configured to generate pressure data. The pressure sensors can be located on or near a lens of the ultrasound scanner, and indicate when the ultrasound scanner is pressed against a patient. The ultrasound system can generate the request based on the pressure data. For instance, the ultrasound system can compare the pressure data to a threshold pressure, and generate the request based on the comparison, e.g., when the pressure data indicates a larger pressure than the threshold pressure according to the comparison.

Additionally or alternatively, the ultrasound scanner can include in, under, or on a surface, any suitable type of sensors for determining a grip orientation. In one example, the ultrasound scanner includes capacitive sensors that can measure a capacitance, or change in capacitance, caused by a user's touch or proximity of touch, as is common in touchscreen technologies. Additionally or alternatively, the ultrasound scanner can include pressure sensors configured to determine an amount of pressure caused by the user's grip on the scanner. Hence, the pressure data can indicate a grip orientation on the ultrasound scanner, and the ultrasound system can generate the request based on the grip orientation, e.g., the grip orientation can indicate an intention to use the ultrasound scanner by a clinician gripping the ultrasound scanner.

Additionally or alternatively, the ultrasound system includes one or more location sensors (e.g., cameras, LIDAR for detection and ranging, etc.) implemented to determine a proximity of the ultrasound scanner to a patient. The ultrasound system can generate the request based on the proximity, such as when the proximity is less than a threshold proximity.

In some embodiments, the ultrasound scanner includes one or more buttons or switches implemented to receive the request as a user input. Additionally or alternatively, in some embodiments, the ultrasound system includes a voice recognition circuit implemented to receive the request as a spoken command.

In some embodiments, the two or more arrays of transducer elements make up a symmetric array architecture having a center row of the transducer elements and a same number of outer rows of the transducer elements on opposing sides of the center row. In some embodiments, the transducer elements of the center row and the outer rows have a common width. In some other embodiments, the transducer elements of the center row and the outer rows have different widths. In yet some other embodiments, the transducer elements of the center row and the outer rows have different operation frequencies and bandwidths.

In some embodiments, the two or more arrays of transducer elements make up an asymmetric array architecture having a center row of the transducer elements and different numbers of outer rows of the transducer elements on opposing sides of the center row.

In some embodiments, the two or more arrays of transducer elements are arranged in concentric shapes. For example, the concentric shapes can include concentric annuli or ellipses. In an example, the concentric shapes include concentric polygons. In another example, the concentric shapes include nested open shapes, such as in the shape of a “V”, “L”, or “C”.

### AN EXAMPLE DEVICE

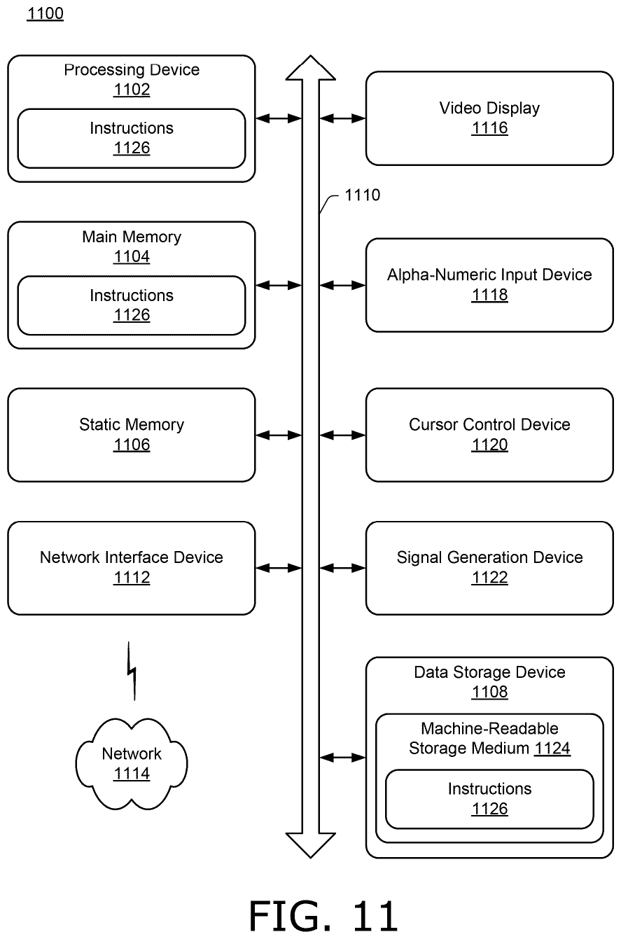

FIG. 11 illustrates a block diagram of an example computing device 1100 that can perform one or more of the operations described herein, in accordance with some implementations. The computing device 1100 can be connected to other computing devices in a local area network (LAN), an intranet, an extranet, and/or the Internet. The computing device can operate in the capacity of a server machine in a client-server network environment or in the capacity of a client in a peer-to-peer network environment. The computing device can be provided by a personal computer (PC), a server computer, a desktop computer, a laptop computer, a tablet computer, a smartphone, an ultrasound machine, or any machine capable of executing a set of instructions (sequential or otherwise) that specify actions to be taken by that machine. Further, while only a single computing device is illustrated, the term “computing device” shall also be taken to include any collection of computing devices that individually or jointly execute a set (or multiple sets) of instructions to perform the methods discussed herein. In some embodiments, the computing device 1100 is one or more of an ultrasound machine, an ultrasound scanner, a docking station, a mobile computing device, a display device, an access point, and a packet-forwarding component.

The example computing device 1100 can include a processing device 1102 (e.g., a general-purpose processor, a programmable logic device (PLD), etc.), a main memory 1104 (e.g., synchronous dynamic random-access memory (DRAM), read-only memory (ROM), etc.), and a static memory 1106 (e.g., flash memory, a data storage device 1108, etc.), which can communicate with each other via a bus 1110. The processing device 1102 can be provided by one or more general-purpose processing devices such as a microprocessor, a central processing unit, or the like. In an illustrative example, the processing device 1102 comprises a complex instruction set computing (CISC) microprocessor, a reduced instruction set computing (RISC) microprocessor, a very long instruction word (VLIW) microprocessor, or a processor implementing other instruction sets or processors implementing a combination of instruction sets. The processing device 1102 can also comprise one or more special-purpose processing devices such as an application-specific integrated circuit (ASIC), a field programmable gate array (FPGA), a digital signal processor (DSP), a network processor, or the like. The processing device 1102 can be configured to execute the operations described herein, in accordance with one or more aspects of the present disclosure, for performing the operations and steps discussed herein.

The computing device 1100 can further include a network interface device 1112, which can communicate with a network 1114. The computing device 1100 also can include a video display unit 1116 (e.g., a liquid crystal display (LCD), an organic light-emitting diode (OLED), a cathode ray tube (CRT), etc.), an alphanumeric input device 1118 (e.g., a keyboard), a cursor control device 1120 (e.g., a mouse), and an acoustic signal generation device 1122 (e.g., a speaker, a microphone, etc.). In some embodiments, the video display unit 1116, the alphanumeric input device 1118, and the cursor control device 1120 can be combined into a single component or device (e.g., an LCD touch screen).

The data storage device 1108 can include a computer-readable storage medium 1124 on which can be stored one or more sets of instructions 1126 (e.g., instructions for carrying out the operations described herein, in accordance with one or more aspects of the present disclosure). The instructions 1126 can also reside, completely or at least partially, within the main memory 1104 and/or within the processing device 1102 during execution thereof by the computing device 1100, where the main memory 1104 and the processing device 1102 also constitute computer-readable media. The instructions can further be transmitted or received over the network 1114 via the network interface device 1112.

Various techniques are described in the general context of software, hardware elements, or program modules. Generally, such modules include routines, programs, objects, elements, components, data structures, and so forth that perform particular tasks or implement particular abstract data types. The terms “module,” “functionality,” and “component” as used herein generally represent software, firmware, hardware, or a combination thereof. In some aspects, the modules described herein are embodied in the data storage device 1108 of the computing device 1100 as executable instructions or code. Although represented as software implementations, the described modules can be implemented as any form of a control application, software application, signal-processing and control module, hardware, or firmware installed on the computing device 1100.

While the computer-readable storage medium 1124 is shown in an illustrative example to be a single medium, the term “computer-readable storage medium” should be taken to include a single medium or multiple media (e.g., a centralized or distributed database and/or associated caches and servers) that store the one or more sets of instructions. The term “computer-readable storage medium” shall also be taken to include any medium that is capable of storing, encoding, or carrying a set of instructions for execution by the machine and that causes the machine to perform the methods described herein. The term “computer-readable storage medium” shall accordingly be taken to include, but not be limited to, solid-state memories, optical media, and magnetic media.

### EXAMPLE ENVIRONMENT

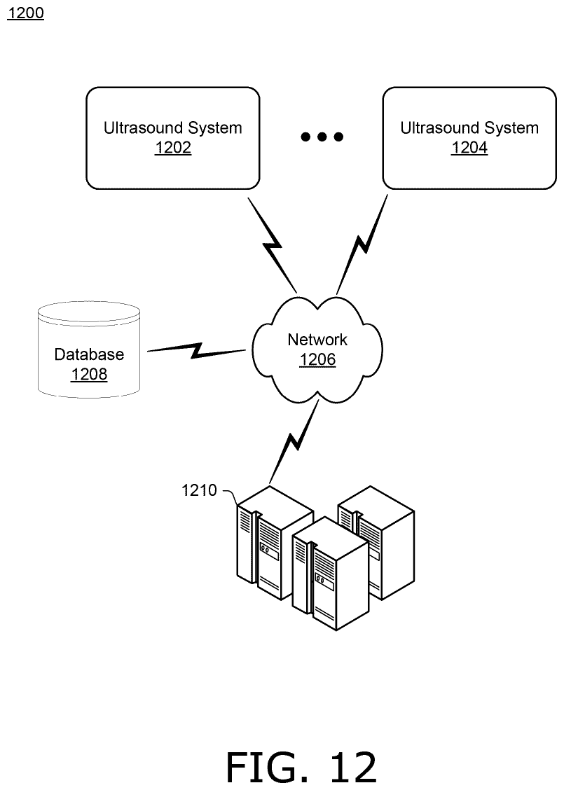

FIG. 12 illustrates an environment 1200 for an ultrasound system in accordance with some embodiments. The environment 1200 includes an ultrasound system 1202 and an ultrasound system 1204. Two example ultrasound systems 1202 and 1204 are illustrated in FIG. 12 for clarity. However, the environment 1200 can include any suitable number of ultrasound systems, such as the ultrasound systems maintained by a care facility or the department of a care facility. Generally, an ultrasound system can include any suitable device (e.g., a component of an ultrasound system). Examples devices of the ultrasound systems 1202 and 1204 include a charging station, an ultrasound machine, a display device (e.g., a tablet or smartphone), an ultrasound scanner, an ultrasound cart, and a display device. Other examples include a transducer cable, a transducer cable holder, a docking station, a scanner station configured to hold one or more ultrasound scanners, a needle guide, a battery for a wireless ultrasound scanner, a battery for an ultrasound machine, a registration system, and the like. The ultrasound systems 1202 and 1204 can include an ultraportable ultrasound system.

The ultrasound systems 1202 and 1204 can be in communication via the network 1206 as part of the environment 1200. The network 1206 can include any suitable network, such as a local area network, a wide area network, a near field communication network, the Internet, an intranet, an extranet, a system bus that couples devices or device components (e.g., in an ASIC, FPGA, or SOC), and combinations thereof. Accordingly, in embodiments, information can be communicated to the ultrasound systems 1202 and 1204 through the network 1206. For instance, the database 1208 can store instructions executable by a processor system of the ultrasound systems 1202 and 1204, and communicate the instructions via the network 1206. The database 1208 can store ultrasound resources and user interface components and share them with the ultrasound systems 1202 and 1204.

The environment 1200 also includes a server system 1210 that can implement any of the functions described herein. The server system 1210 can be a separate device from the ultrasound systems 1202 and 1204. Alternatively, the server system 1210 can be included in at least one of the ultrasound systems 1202 and 1204. In one example, the server system 1210 and the database 1208 are included in at least one of the ultrasound systems 1202 and 1204. In an example, the server system 1210 is implemented as a remote server system that is remote from (e.g., not collocated with) the ultrasound systems 1202 and 1204.

### AN EXAMPLE USER INTERFACE

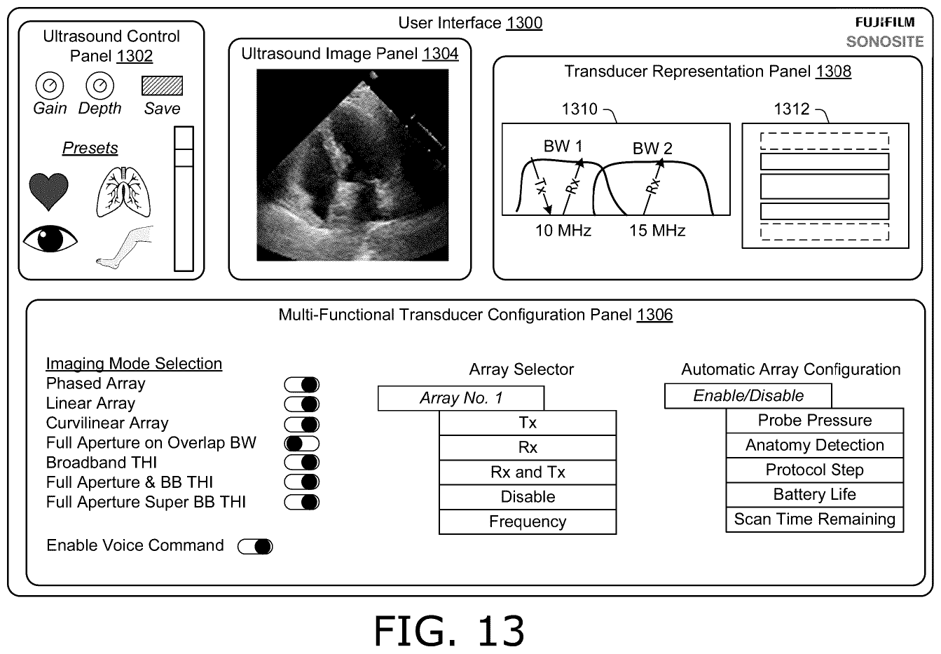

FIG. 13 illustrates an example user interface 1300 of an ultrasound system for controlling a multi-functional transducer in accordance with some embodiments. The user interface 1300 can be displayed by any suitable component of an ultrasound system, including an ultrasound machine, a display device, a mobile computing device, an ultrasound machine, an ultrasound scanner, etc. In some embodiments, the user interface 1300 includes an ultrasound control panel 1302, an ultrasound image panel 1304, a multi-functional transducer configuration panel 1306, and a transducer representation panel 1308. These panels are illustrated as separate (e.g., distinct) panels in FIG. 13 for clarity as example user interface components, and are not meant to be limiting. These panels can be combined, separated, resized, reshaped and reorganized in any suitable way.

In some embodiments, the ultrasound control panel 1302 includes any suitable controls and settings for controlling an ultrasound system, such as depth and gain adjustments, and a button to store/save images and/or video clips. The ultrasound control panel 1302 can also include icons to select examination presets, such as a heart icon for a cardiac preset, a lung icon for a respiratory preset, an eye icon for an ocular present, and a leg icon for a muscular-skeletal preset. The ultrasound control panel 1302 can also include options (not illustrated in FIG. 13) to enable one or more neural networks for processing of an ultrasound image, such as an ultrasound image displayed in the ultrasound image panel 1304. For example, a cardiac neural network can be enabled to generate a value of ejection fraction, a free fluid network can be enabled to generate a segmentation of free fluid in an ultrasound image, and a pneumothorax (PTX) neural network can be enabled to generate a probability of a pneumothorax condition or collapsed lung.

The ultrasound image panel 1304 can display any suitable ultrasound image, such as a B-mode image, M-mode image, Doppler image, etc. The ultrasound image panel 1304 can also display a measurement, annotation, classification, and the like. In embodiments, the ultrasound image panel 1304 can display an inference generated by a neural network, such as a segmentation of a patient anatomy.

In some embodiments, the multi-functional transducer configuration panel 1306 includes controls for enabling and disabling arrays of a multi-functional transducer and/or setting imaging modes for imaging with a multi-functional transducer. The multi-functional transducer configuration panel 1306 in FIG. 13 includes switches to enable a phased array, a linear array, and a curvilinear array. In some embodiments, the multi-functional transducer configuration panel 1306 also includes switches for selecting imaging modes including a full aperture on overlap bandwidth mode (which is selected in the example in FIG. 13), a broadband THI mode, a full aperture and broadband THI mode, and a full aperture super broadband THI mode. Examples of these imaging modes were previously discussed with respect to FIG. 9. The multi-functional transducer configuration panel 1306 also includes a switch to enable the use of voice command to control the multi-functional transducer.

In some embodiments, the multi-functional transducer configuration panel 1306 also includes pull-down tabs to select an array and configure the array. For example, the multi-functional transducer configuration panel 1306 in FIG. 13 includes an array selector in which “Array No. 1” has been selected, and various pull-down options that can be selected to configure this array, including to enable the array for transmission only, reception only, transmission and reception, and to disable the array. In some embodiments, the pull-down options also include a selection of a frequency for the array (e.g., an ultrasound frequency or center frequency). Although not shown for clarity, the pull-down options can also include one more selections for setting an array bandwidth, such as a bandwidth span in hertz, or selections of low, medium, and high bandwidth configurations.

In some embodiments, the multi-functional transducer configuration panel 1306 also includes pull-down tabs to enable the multi-functional transducer in an automatic mode that automatically configures the arrays based on a determination made by the ultrasound system. Example determinations displayed in FIG. 13 that can be used to automatically configure the arrays include selections for probe pressure, anatomy detection, protocol step, battery life, and scan time remaining. In some embodiments, a user can program for each of these selections how to configure the arrays of the multi-functional transducer. As an example, the “probe pressure” selection can be mapped to an imaging mode of full aperture on overlap bandwidth, so that when the probe pressure exceeds a threshold probe pressure, the multi-functional transducer is automatically configured to image according to the mode of full aperture on overlap bandwidth. In another example, the “battery life remaining” selection can be mapped to an imaging mode that uses only a linear array, so that when the battery life drops below a threshold amount, the ultrasound scanner switches to imaging with a linear array only (and disables other arrays) to conserve power.

The transducer representation panel 1308 can display any suitable type and number of visual representations for a multi-functional transducer that indicates an imaging mode and/or array configuration for elements of arrays of the multi-functional transducer. In FIG. 13, the transducer representation panel 1308 includes an exemplary visual representation 1310 that displays bandwidths for two arrays, BW1 and BW2 graphically and with text of 10 MHz and 15 MHz, respectively. In some embodiments, the visual representation 1310 includes an arrow with a “Tx” label for transmission and an arrow with an “Rx” label for reception for the first array with bandwidth BW1, indicating that the first array is used for both transmission and reception of ultrasound. In some embodiments, the visual representation 1310 also includes an arrow with an arrow with an “Rx” label for reception for the second array with bandwidth BW2, indicating that the second array is used for reception of ultrasound (and not transmission).

In some embodiments, the transducer representation panel 1308 in FIG. 13 also includes an exemplary visual representation 1312 for a multi-functional transducer having first, second, and third arrays. The first array comprises a center arrow of elements. The second array comprises two side arrays that are adjacent to the first array. In some embodiments, the third array comprises two outer arrays that are adjacent to the rows of the second array. In some embodiments, the visual representation 1312 displays the first array and the second array as solid lines to indicate that these arrays of the multi-functional transducer are active/enabled. In some embodiments, the visual representation 1312 also displays the third array with dashed lines to indicate that the rows of the third array of the multi-functional transducer are off/disabled.

The visual representations 1310 and 1312 are meant to be exemplary and non-limiting. In some embodiments, a user can select what visual representations are displayed in transducer representation panel 1308, such as with pull-down tabs (not shown for clarity). In some embodiments, the transducer representation panel 1308 updates the visual representations it displays based on the imaging mode and/or array configuration selected in the multi-functional transducer configuration panel 1306.

### Additional Exemplary Array Configurations

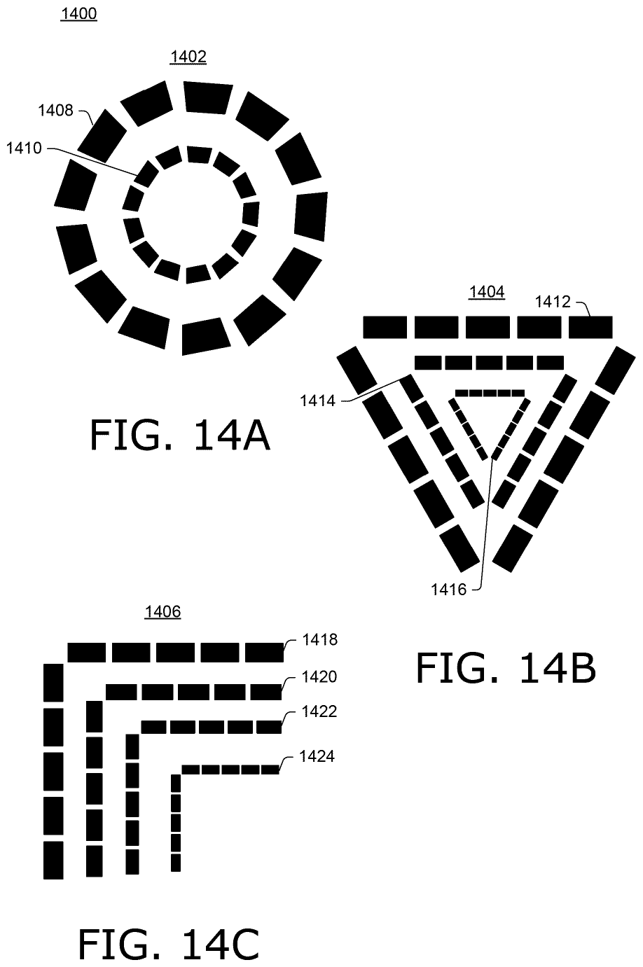

The discussions of arrays of a multi-functional transducer above largely focus on arrays comprised of rows of transducer elements, as illustrated in FIGS. 3A-5B. However, the techniques disclosed herein are not limited to rows of transducer elements as previously described, but can also include multiple arrays in various configurations. For example, FIGS. 14A-C include multi-dimensional array architectures 1400 in accordance with some embodiments. The multi-dimensional array architectures 1400 include a circular array 1402, a polygonal array 1404, and an open-shaped array 1406.

In some embodiments, the circular array 1402 includes an outer array 1408 of transducer elements and an inner array 1410 of transducer elements arranged in concentric circles. Although circles are illustrated in the circular array 1402, the outer array 1408 and the inner array 1410 can include elements arranged in concentric ellipses in embodiments.

In some embodiments, the polygonal array 1404 includes three nested arrays of triangular shape, including an outer array 1412 of transducer elements, a center array 1414 of transducer elements, and an inner array 1416 of transducer elements. The triangular shapes of the three arrays of the polygonal array 1404 are examples of polygons and are meant to be exemplary. Other polygonal shapes that can be included in the polygonal array 1404 include nested arrays arranged in rectangular, rhombus, pentagon, and the like shapes.

In some embodiments, the open-shaped array 1406 includes four nested arrays of L-shapes, including a first outer array 1418 of transducer elements, a second outer array 1420 of transducer elements, a first inner array 1422 of transducer elements, and a second inner array 1424 of transducer elements. The L-shapes of the four arrays of the open-shaped array 1406 are examples of open shapes and are meant to be exemplary. Other open shapes that can be included in the open-shaped array 1406 include nested arrays arranged in C-shapes, V-shapes, S-shapes, and the like.

As discussed above with respect to the rows of array elements, e.g., with regards to FIGS. 3A-5B, the arrays of the multi-dimensional array architectures 1400 can have different or the same frequencies, different or the same bandwidths, and/or different or the same widths.

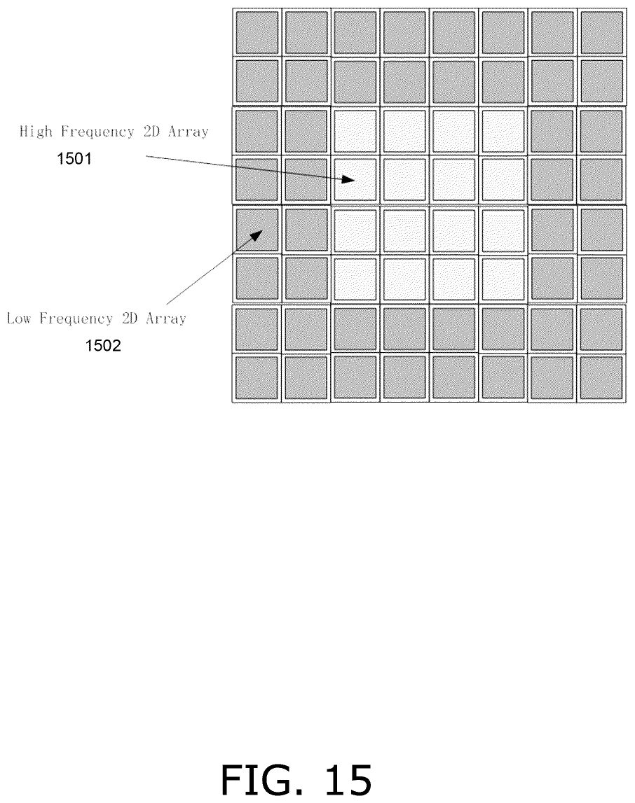

FIG. 15 illustrates some embodiments of multi-dimensional array architectures. Referring to FIG. 15, a high frequency 2D array 1501 of transducer is in the centrally located within a low frequency 2D array 1502 of transducer elements. High frequency 2D array 1501 is 4×4 square array and low frequency 2D array 1502 comprises two rows of elements around high frequency 2D array 1501 in a ring arrangement (e.g., 2 rings of elements around high frequency 2D array 1501). Note that a high frequency 2D array 1501 and low frequency 2D array 1502 are not limited to these numbers of elements and rows. Furthermore, there can be more (or less) rings of low frequency 2D array 1502 around high frequency 2D array 1501. Also, in some other embodiments, there are multiple rings of transducer elements of different frequencies around a central array high frequency transducer elements. In some other embodiments, low frequency 2D array 1502 of transducer elements is centrally located with one or more rings of transducer elements of high frequency 2D array 1501 encircling it.

There is a number of example embodiments described herein.

Example 1 is an ultrasound device comprising: a lens; an array coupled to the lens and having a center row of transducer elements of a first width that operate at a first frequency and two or more outer rows of transducer elements of a second or other widths that operate at a second or other frequencies different than the first frequency, wherein the first width is different than the second width, the center row being between the two or more outer rows; and a controller coupled to the array and configured to control the center row of transducer elements and two or more outer rows of transducer elements to operate at a same time or at different times.

Example 2 is the ultrasound device of example 1 that may optionally include that, during operation, the center row operates as a linear array and a pair of rows of the two or more rows operate as a phased array.

Example 3 is the ultrasound device of example 2 that may optionally include that the first frequency is twice the second frequency.

Example 4 is the ultrasound device of example 3 that may optionally include that element pitches for both center row and the pair of rows are equal.

Example 5 is the ultrasound device of example 1 that may optionally include that the controller controls the array in a plurality of modes in which either the center row of transducer elements or the two or more rows of transducer elements is operating or both the center row of transducer elements and the two or more rows of transducer elements are operating at the same time based on which mode of the plurality of modes is being used.

Example 6 is the ultrasound device of example 5 that may optionally include that the controller is configured to control the center row of transducer elements and two or more outer rows of transducer elements independently in one of the modes to operate at the same time to obtain signals for performing super broadband harmonic imaging by controlling the center row of transducer elements to perform a receive operation while controlling the two or more rows of transducer elements to perform transmit operations.

Example 7 is the ultrasound device of example 5 that may optionally include that the plurality of modes includes: a first mode in which the controller causes both the center row and the two or more rows of transducer elements to perform both transmit and receive operations to provide signals for full aperture imaging on an area of bandwidth produced by both that overlaps; a second mode in which the controller causes the center row of transducer elements to perform a receive operation and the two or more rows of transducer elements to perform transmit and receive operations to provide signals for Tissue Harmonic Imaging (THI); and a third mode in which the controller causes both the center row and the two or more rows of transducer elements to perform both transmit and receive operations to provide signals for super broadband full aperture THI.

Example 8 is the ultrasound device of example 5 that may optionally include a user interface to enable a user to cause the controller to switch between modes of the plurality of modes.

Example 9 is the ultrasound device of example 8 that may optionally include a probe enclosure that contains the array, and wherein the user interface comprises one or more buttons, one or more sensors, one or more switches coupled to the probe enclosure, or voice control to switch between the modes.

Example 10 is the ultrasound device of example 8 that may optionally include that the user interface comprises one or more buttons, one or more sensors, or one or more switches coupled to an ultrasound system or voice control communicably coupled to the array to switch between the modes.

Example 11 is the ultrasound device of example 1 that may optionally include that each of the transducer elements is part of an acoustic stack that includes a backing block through which at least a signal coupled to said each transducer element traverses.

Example 12 is the ultrasound device of example 1 that may optionally include that each of the transducer elements is part of an acoustic stack, and stacks associated with transducer elements of the center row have different focal depths and beam characteristics than stacks associated with transducer elements of the two or more rows.

Example 13 is the ultrasound device of example 1 that may optionally include one or more system connectors to interface signals from each of the transducer elements of the center row of transducer elements and the two or more rows of transducer elements to an ultrasound system, the one or more system connectors comprising first and second sets of tuning inductors, the first set of tuning inductors for interfacing signals from the transducer elements of the center row of transducer elements and the second set of tuning inductors for interfacing signals from the transducer elements of the two or more rows of transducer elements using tuning inductors with different inductor values than tuning inductors of the first set of tuning inductors.

Example 14 is the ultrasound device of example 1 that may optionally include that each of the transducer elements comprises a piezoelectric element.

Example 15 is an ultrasound device comprising: a lens; a high frequency array comprising a center row of transducer elements that operate at a first frequency and a low frequency array comprising two outer rows of transducer elements that operate at a second or other frequencies different than the first frequency, the center row of transducer elements being between the two outer rows of transducer elements; and a controller coupled and configured to control each of the high frequency array and the low frequency array in a plurality of modes to operate at a same time or at different times. The modes include a first mode in which the controller controls the high frequency array to operate as a linear array while low frequency array is not operating; a second mode in which the controller controls the low frequency array to operate as a phased array while the high frequency array is not operating; and a third mode in which the controller controls the high and low frequency arrays to operate at the same time to obtain signals for performing super broadband harmonic imaging.

Example 16 is the ultrasound device of example 15 that may optionally include that, during the third mode, the high frequency array filter out reflections from transmitted signals from the low frequency array.

Example 17 is the ultrasound device of example 15 that may optionally include that the plurality of modes includes one or more of: a fourth mode in which the controller causes both the high and low frequency arrays to perform both transmit and receive operations to provide signals for full aperture imaging on an area of bandwidth produced by both that overlaps; a fifth mode in which the controller causes the high frequency array to perform a receive operation and the low frequency array to perform both transmit and receive operations to provide signals for Tissue Harmonic Imaging (THI); a sixth mode in which the controller causes both the high and low frequency arrays to perform both transmit and receive operations to provide signals for super broadband full aperture THI.

Example 18 is the ultrasound device of example 15 that may optionally include that the first frequency is twice the second frequency, and wherein element pitches for both high frequency array and the low frequency array are equal.

Example 19 is method of controlling an ultrasound system, the method comprising: controlling a multi-frequency and multi-dimensional array of transducer elements having a high frequency array comprising a center row of transducer elements that operate at a first frequency and a low frequency array comprising two outer rows of transducer elements that operate at a second or other frequencies different than the first frequency, the center row of transducer elements being between the two outer rows of transducer elements, wherein controlling the multi-frequency and multi-dimensional array comprises controlling both the high frequency array and low frequency array to operate at the same time; receiving, by both the high and low frequency arrays, reflected signals; and performing super broadband harmonic imaging based on the received signals.

Example 20 is the method of example 19 that may optionally include that bandwidth of the received signals covers bandwidth across both the high and low frequency arrays.

All of the methods and tasks described herein may be performed and fully automated by a computer system. The computer system may, in some cases, include multiple distinct computers or computing devices (e.g., physical servers, workstations, storage arrays, cloud computing resources, etc.) that communicate and interoperate over a network to perform the described functions. Each such computing device typically includes a processor (or multiple processors) that executes program instructions or modules stored in a memory or other non-transitory computer-readable storage medium or device (e.g., solid state storage devices, disk drives, etc.). The various functions disclosed herein may be embodied in such program instructions or may be implemented in application-specific circuitry (e.g., ASICs or FPGAs) of the computer system. Where the computer system includes multiple computing devices, these devices may, but need not, be co-located. The results of the disclosed methods and tasks may be persistently stored by transforming physical storage devices, such as solid-state memory chips or magnetic disks, into a different state. In some embodiments, the computer system may be a cloud-based computing system whose processing resources are shared by multiple distinct business entities or other users.

Depending on the embodiment, certain acts, events, or functions of any of the processes or algorithms described herein can be performed in a different sequence, can be added, merged, or left out altogether (e.g., not all described operations or events are necessary for the practice of the algorithm). Moreover, in some embodiments, operations or events can be performed concurrently, e.g., through multi-threaded processing, interrupt processing, or multiple processors or processor cores or on other parallel architectures, rather than sequentially.

The various illustrative logical blocks, modules, routines, and algorithm steps described in connection with the embodiments disclosed herein can be implemented as electronic hardware (e.g., ASICs or FPGA devices), computer software that runs on computer hardware, or combinations of both. Moreover, the various illustrative logical blocks and modules described in connection with the embodiments disclosed herein can be implemented or performed by a machine, such as a processor device, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor device can be a microprocessor, but in the alternative, the processor device can be a controller, microcontroller, or state machine, combinations of the same, or the like. A processor device can include electrical circuitry configured to process computer-executable instructions. In another embodiment, a processor device includes an FPGA or other programmable device that performs logic operations without processing computer-executable instructions. A processor device can also be implemented as a combination of computing devices, e.g., a combination of a DSP and a microprocessor, a plurality of microprocessors, one or more microprocessors in conjunction with a DSP core, or any other such configuration. Although described herein primarily with respect to digital technology, a processor device may also include primarily analog components. For example, some or all of the rendering techniques described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

The elements of a method, process, routine, or algorithm described in connection with the embodiments disclosed herein can be embodied directly in hardware, in a software module executed by a processor device, or in a combination of the two. A software module can reside in RAM memory, flash memory, ROM memory, EPROM memory, EEPROM memory, registers, hard disk, a removable disk, a CD-ROM, or any other form of a non-transitory computer-readable storage medium. An exemplary storage medium can be coupled to the processor device such that the processor device can read information from, and write information to, the storage medium. In the alternative, the storage medium can be integral to the processor device. The processor device and the storage medium can reside in an ASIC. The ASIC can reside in a user terminal. In the alternative, the processor device and the storage medium can reside as discrete components in a user terminal.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements, or steps. Thus, such conditional language is not generally intended to imply that features, elements, or steps are in any way required for one or more embodiments or that one or more embodiments necessarily include logic for deciding, with or without other input or prompting, whether these features, elements or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Disjunctive language such as the phrase “at least one of X, Y, or Z,” unless specifically stated otherwise, is otherwise understood with the context as used in general to present that an item, term, etc., may be either X, Y, or Z, or any combination thereof (e.g., X, Y, or Z). Thus, such disjunctive language is not generally intended to, and should not, imply that certain embodiments require at least one of X, at least one of Y, and at least one of Z to each be present.

While the above detailed description has shown, described, and pointed out novel features as applied to various embodiments, it can be understood that various omissions, substitutions, and changes in the form and details of the devices or algorithms illustrated can be made without departing from the spirit of the disclosure. As can be recognized, certain embodiments described herein can be embodied within a form that does not provide all of the features and benefits set forth herein, as some features can be used or practiced separately from others. The scope of certain embodiments disclosed herein is indicated by the appended claims rather than by the foregoing description. All changes which come within the meaning and range of equivalency of the claims are to be embraced within their scope.

We claim:

1. An ultrasound device comprising:
a lens;
an array coupled to the lens and having a center row of transducer elements of a first width that operate at a first frequency and two or more outer rows of transducer elements of a second or other widths that operate at a second or other frequencies different than the first frequency, wherein the first width is different than the second width, the center row being between the two or more outer rows; and
a controller coupled to the array and configured to control the center row of transducer elements and two or more outer rows of transducer elements to operate at a same time or at different times.

2. The ultrasound device of claim 1 wherein, during operation, the center row operates as a linear array and a pair of rows of the two or more rows operate as a phased array.

3. The ultrasound device of claim 2 wherein the first frequency is twice the second frequency.

4. The ultrasound device of claim 3 wherein element pitches for both center row and the pair of rows are equal.

5. The ultrasound device of claim 1 wherein the controller controls the array in a plurality of modes in which either the center row of transducer elements or the two or more rows of transducer elements is operating or both the center row of transducer elements and the two or more rows of transducer elements are operating at the same time based on which mode of the plurality of modes is being used.

6. The ultrasound device of claim 5 wherein the controller is configured to control the center row of transducer elements and two or more outer rows of transducer elements independently in one of the modes to operate at the same time to obtain signals for performing super broadband harmonic imaging by controlling the center row of transducer elements to perform a receive operation while controlling the two or more rows of transducer elements to perform transmit operations.

7. The ultrasound device of claim 5 wherein the plurality of modes includes:
a first mode in which the controller causes both the center row and the two or more rows of transducer elements to perform both transmit and receive operations to provide signals for full aperture imaging on an area of bandwidth produced by both that overlaps;
a second mode in which the controller causes the center row of transducer elements to perform a receive operation and the two or more rows of transducer elements to perform transmit and receive operations to provide signals for Tissue Harmonic Imaging (THI);
a third mode in which the controller causes both the center row and the two or more rows of transducer elements to perform both transmit and receive operations to provide signals for super broadband full aperture THI.

8. The ultrasound device of claim 5 further comprising a user interface to enable a user to cause the controller to switch between modes of the plurality of modes.

9. The ultrasound device of claim 8 further comprising a probe enclosure that contains the array, and wherein the user interface comprises one or more buttons, one or more sensors, one or more switches coupled to the probe enclosure, or voice control to switch between the modes.

10. The ultrasound device of claim 8 wherein the user interface comprises one or more buttons, one or more sensors, or one or more switches coupled to an ultrasound system or voice control communicably coupled to the array to switch between the modes.

11. The ultrasound device of claim 1 wherein each of the transducer elements is part of an acoustic stack that includes a backing block through which at least a signal coupled to said each transducer element traverses.

12. The ultrasound device of claim 1 wherein each of the transducer elements is part of an acoustic stack, and stacks associated with transducer elements of the center row have different focal depths and beam characteristics than stacks associated with transducer elements of the two or more rows.

13. The ultrasound device of claim 1 further comprising one or more system connectors to interface signals from each of the transducer elements of the center row of transducer elements and the two or more rows of transducer elements to an ultrasound system, the one or more system connectors comprising first and second sets of tuning inductors, the first set of tuning inductors for interfacing signals from the transducer elements of the center row of transducer elements and the second set of tuning inductors for interfacing signals from the transducer elements of the two or more rows of transducer elements using tuning inductors with different inductor values than tuning inductors of the first set of tuning inductors.

14. The ultrasound device of claim 1 wherein each of the transducer elements comprises a piezoelectric element.

15. An ultrasound device comprising:
a lens;
a high frequency array comprising a center row of transducer elements that operate at a first frequency and a low frequency array comprising two outer rows of transducer elements that operate at a second or other frequencies different than the first frequency, the center row of transducer elements being between the two outer rows of transducer elements; and
a controller coupled and configured to control each of the high frequency array and the low frequency array in a plurality of modes to operate at a same time or at different times, including
a first mode in which the controller controls the high frequency array to operate as a linear array while low frequency array is not operating;
a second mode in which the controller controls the low frequency array to operate as a phased array while the high frequency array is not operating; and
a third mode in which the controller controls the high and low frequency arrays to operate at the same time to obtain signals for performing super harmonic broadband imaging.

16. The ultrasound device of claim 15 wherein, during the third mode, the high frequency array filter out reflections from transmitted signals from the low frequency array.

17. The ultrasound device of claim 15 wherein the plurality of modes includes one or more of:
a fourth mode in which the controller causes both the high and low frequency arrays to perform both transmit and receive operations to provide signals for full aperture imaging on an area of bandwidth produced by both that overlaps;
a fifth mode in which the controller causes the high frequency array to perform a receive operation and the low frequency array to perform both transmit and receive operations to provide signals for Tissue Harmonic Imaging (THI);
a sixth mode in which the controller causes both the high and low frequency arrays to perform both transmit and receive operations to provide signals for super broadband full aperture THI.

18. The ultrasound device of claim 15 wherein the first frequency is twice the second frequency, and wherein element pitches for both high frequency array and the low frequency array are equal.

19. A method of controlling an ultrasound system, the method comprising:
controlling a multi-frequency and multi-dimensional array of transducer elements having a high frequency array comprising a center row of transducer elements that operate at a first frequency and a low frequency array comprising two outer rows of transducer elements that operate at a second or other frequencies different than the first frequency, the center row of transducer elements being between the two outer rows of transducer elements, wherein controlling the multi-frequency and multi-dimensional array comprises controlling both the high frequency array and low frequency array to operate at the same time; and
receiving, by both the high and low frequency arrays, reflected signals; and
performing super broadband harmonic imaging based on the received signals.

20. The method of claim 19 wherein bandwidth of the received signals covers bandwidth across both the high and low frequency arrays.
